(19) United States
(12) Patent Application Publication
(10) Pub. No.: US 2010/0324036 A1
(43) Pub. Date: Dec. 23, 2010

(54) SUBSTITUTED IMIDAZOPYRIDINE DERIVATIVES AS MELANOCORTIN-4 RECEPTOR ANTAGONISTS

(75) Inventors: Michael Soeberdt, Rheinfelden (DE); Philipp Weyermann, Sissach (CH); Herve Siendt, Ranspach-le-Haut (FR); Sonja Nordhoff, Arlesheim (CH); Achim Feurer, Auggen (DE); Miroslav Terinek, Bubendorf (CH)

Correspondence Address: Workman Nydegger;1000 Eagle Gate Tower, 60 East South Temple, Salt Lake City, UT, 84111, US

(73) Assignee: SANTHERA PHARMACEUTICALS (SCHWEIZ) AG, LIESTAL (CH)

(21) Appl. No.: 12/809,824

(22) PCT Filed: Dec. 18, 2008
(86) PCT No.: PCT/EP2008/010849

§ 371 Date: Sep. 2, 2010

Related U.S. Application Data
(60) Provisional application No. 61/016,007, filed on Dec. 21, 2007.

(30) Foreign Application Priority Data
Dec. 21, 2007 (EP) 07025029.5

(51) Int. Cl. A61K 31/5383; C07D 471/04; A61K 31/437; C07D 498/04; A61P 9/00; A61P 21/00; A61P 25/00; A61P 25/22; A61P 25/24; A61P 13/12; A61P 35/00; A61P 29/00

(52) U.S. Cl. 514/230.5; 546/121; 514/300; 544/105

(57) ABSTRACT

The present invention relates to substituted imidazopyridine derivatives as melanocortin-4 receptor (MC-4R) modulators, in particular as melanocortin 4 receptor antagonists. The antagonists are useful for the treatment of disorders and diseases such as cachexia induced by e.g. cancer, chronic kidney disease (CKD) or chronic heart failure (CHF), muscle wasting, anorexia induced by e.g. chemotherapy or radiotherapy, anorexia nervosa, amyotrophic lateral sclerosis (ALS), pain, neuropathic pain, anxiety and depression.

### FIELD OF THE INVENTION

The present invention relates to substituted imidazopyridine derivatives as melanocortin-4 receptor modulators. Depending on the structure and the stereochemistry, melanocortin-4 receptor modulators are either agonists or antagonists. The compounds of the invention are selective antagonists of the human melanocortin-4 receptor (MC-4R). The antagonists are useful for the treatment of disorders and diseases such as cachexia induced by e.g. cancer, chronic kidney disease (CKD) or chronic heart failure (CHF), muscle wasting, anorexia induced by e.g. chemotherapy or radiotherapy, anorexia nervosa, amyotrophic lateral sclerosis (ALS), pain, neuropathic pain, anxiety and depression.

### BACKGROUND OF THE INVENTION

Melanocortins (MCs) stem from pro-opiomelanocortin (POMC) via proteolytic cleavage. These peptides, adrenocorticotropic hormone (ACTH), α-melanocyte-stimulating hormone (α-MSH), β-MSH and γ-MSH, range in size from 12 to 39 amino acids. The most important endogenous agonist for central MC-4R activation appears to be the tridecapeptide α-MSH. Among MCs, it was reported that α-MSH acts as a neurotransmitter or neuromodulator in the brain. MC peptides, particularly α-MSH, have a wide range of effects on biological functions including feeding behavior, pigmentation and exocrine function. The biological effects of α-MSH are mediated by a sub-family of 7-transmembrane G-protein-coupled receptors, termed melanocortin receptors (MC-R5). Activation of any of these MC-R5 results in stimulation of cAMP formation.

To date, five distinct types of receptor subtype for MC (MC-1R to MC-5R) have been identified and these are expressed in different tissues.

MC-1R was first found in melanocytes. Naturally occurring inactive variants of MC-1R in animals were shown to lead to alterations in pigmentation and a subsequent lighter coat color by controlling the conversion of phaeomelanin to eumelanin through the control of tyrosinase. From these and other studies, it is evident that MC-1R is an important regulator of melanin production and coat color in animals and skin color in humans. The MC-2R is expressed in the adrenal gland representing the ACTH receptor. The MC-2R is not a receptor for α-MSH but is the receptor for the adrenocorticotropic hormone I (ACTH I).

The MC-3R is expressed in the brain (predominately located in the hypothalamus) and peripheral tissues like gut and placenta, and knock-out studies have revealed that the MC-3R may be responsible for alterations in feeding behavior, body weight and thermogenesis.

The MC-4R is primarily expressed in the brain. Overwhelming data support the role of MC-4R in energy homeostasis. Genetic knock-outs and pharmacologic manipulation of MC-4R in animals have shown that agonizing the MC-4R causes weight loss and antagonizing the MC-4R produces weight gain (A. Kask, et al., “Selective antagonist for the melanocortin-4 receptor (HS014) increases food intake in free-feeding rats,” Biochem. Biophys. Res. Commun., 245: 90-93 (1998)).

MC-5R is ubiquitously expressed in many peripheral tissues including white fat, placenta and a low level of expression is also observed in the brain. However its expression is greatest in exocrine glands. Genetic knock-out of this receptor in mice results in altered regulation of exocrine gland function, leading to changes in water repulsion and thermoregulation. MC-5R knockout mice also reveal reduced sebaceous gland lipid production (Chen et al., Cell, 91: 789-798 (1997)).

Attention has been focused on the study of MC-3R and MC-4R modulators and their use in treating body weight disorders, such as obesity and anorexia. However, evidence has shown that the MC peptides have potent physiological effects besides their role in regulating pigmentation, feeding behavior and exocrine function. In particular, α-MSH recently has been shown to induce a potent anti-inflammatory effect in both acute and chronic models of inflammation including inflammatory bowel-disease, renal ischemia/reperfusion injury and endotoxin-induced hepatitis. Administration of α-MSH in these models results in substantial reduction of inflammation-mediated tissue damage, a significant decrease in leukocyte infiltration and a dramatic reduction in elevated levels of cytokines and other mediators to near baseline levels. Recent studies have demonstrated that the anti-inflammatory actions of α-MSH are mediated by MC-1R. The mechanism by which agonism of MC-1R results in an anti-inflammatory response is likely through inhibition of the pro-inflammatory transcription activator, NF-κB. NF-κB is a pivotal component of the pro-inflammatory cascade, and its activation is a central event in initiating many inflammatory diseases. Additionally, anti-inflammatory actions of α-MSH may be, in part, mediated by agonism of MC-3R and/or MC-5R.

A specific single MC-R that may be targeted for the control of obesity has not yet been identified, although evidence has been presented that MC-4R signaling is important in mediating feeding behavior (S. Q. Giraudo et al., “Feeding effects of hypothalamic injection of melanocortin-4 receptor ligands,” Brain Research, 80: 302-306 (1998)). Further evidence for the involvement of MC-R5 in obesity includes: 1) the agouti (A^{vy}) mouse which ectopically expresses an antagonist of the MC-1R, MC-3R and MC-4R is obese, indicating that blocking the action of these three MC-R's can lead to hyperphagia and metabolic disorders; 2) MC-4R knockout mice (D. Huszar et al., Cell, 88: 131-141 (1997)) recapitulate the phenotype of the agouti mouse and these mice are obese; 3) the cyclic heptapeptide melanotanin II (MT-II) (a non-selective MC-1R, -3R, -4R, and -5R agonist) injected intracerebroventricularly (ICV) in rodents, reduces food intake in several animal feeding models (NPY, ob/ob, agouti, fasted) while ICV injected SHU-9119 (MC-3R and 4R antagonist; MC-1R and -5R agonist) reverses this effect and can induce hyperphagia; 4) chronic intraperitoneal treatment of Zucker fatty rats with an α-NDP-MSH derivative (HP-228) has been reported to activate MC-1R, -3R, -4R, and -5R and to attenuate food intake and body weight gain over a 12 week period (I. Corcos et al., “HP-228 is a potent agonist of melanocortin receptor-4 and significantly attenuates obesity and diabetes in Zucker fatty rats,” Society for Neuroscience Abstracts, 23: 673 (1997)).

MC-4R appears to play a role in other physiological functions as well, namely controlling grooming behavior, erection and blood pressure. Erectile dysfunction denotes the medical condition of inability to achieve penile erection sufficient for successful intercourse. The term “impotence” is often employed to describe this prevalent condition. Synthetic melanocortin receptor agonists have been found to initiate erections in men with psychogenic erectile dysfunction (H. Wessells et al., “Synthetic Melanotropic Peptide Initiates Erections in Men With Psychogenic Erectile Dysfunction: Double-Blind, Placebo Controlled Crossover Study,” J. Urol., 160: 389-393, 1998). Activation of melanocortin receptors of the brain appears to cause normal stimulation of sexual arousal. Evidence for the involvement of MC-R in male and/or female sexual dysfunction is detailed in WO 00/74679.

Diabetes is a disease in which a mammal's ability to regulate glucose levels in the blood is impaired because the mammal has a reduced ability to convert glucose to glycogen for storage in muscle and liver cells. In Type I diabetes, this reduced ability to store glucose is caused by reduced insulin production. “Type II diabetes” or “Non-Insulin Dependent Diabetes Mellitus” (NIDDM) is the form of diabetes which is due to a profound resistance to insulin stimulating or regulatory effect on glucose and lipid metabolism in the main insulin-sensitive tissues, muscle, liver and adipose tissue. This resistance to insulin responsiveness results in insufficient insulin activation of glucose uptake, oxidation and storage in muscle, and inadequate insulin repression of lipolysis in adipose tissue and of glucose production and secretion in liver. When these cells become desensitized to insulin, the body tries to compensate by producing abnormally high levels of insulin and hyperinsulemia results. Hyperinsulemia is associated with hypertension and elevated body weight. Since insulin is involved in promoting the cellular uptake of glucose, amino acids and triglycerides from the blood by insulin sensitive cells, insulin insensitivity can result in elevated levels of triglycerides and LDL which are risk factors in cardiovascular diseases. The constellation of symptoms which includes hyperinsulemia combined with hypertension, elevated body weight, elevated triglycerides and elevated LDL, is known as Syndrome X. MC-4R agonists might be useful in the treatment of NIDDM and Syndrome X.

Among MC receptor subtypes, the MC4 receptor is also of interest in terms of the relationship to stress and the regulation of emotional behavior, as based on the following findings. Stress initiates a complex cascade of responses that include endocrine, biochemical and behavioral events. Many of these responses are initiated by release of corticotropin-releasing factor (CRF) (Owen M J and Nemeroff C B (1991) Physiology and pharmacology of corticotrophin releasing factor. Pharmacol Rev 43: 425-473). In addition to activation of the brain CRF system, there are several lines of evidence that melanocortins (MCs), which stem from proopiomelanocortin by enzymatic processing, mediate important behavioral and biochemical responses to stress and, consequently, stress-induced disorders like anxiety and depression (Anxiolytic-Like and Antidepressant-Like Activities of MCL0129 (1-[(S)-2-(4-Fluorophenyl)-2-(4-isopropylpiperadin-1-yl)ethyl]-4-[4-(2-methoxynaphthalen-1-yl)butyl]piperazine), a Novel and Potent Nonpeptide Antagonist of the Melanocortin-4 Receptor; Shigeyuki Chaki et al, J. Pharm. Exp. Ther. (2003) 304(2), 818-26).

Chronic diseases, such as malignant tumors or infections, are frequently associated with cachexia resulting from a combination of a decrease in appetite and a loss of lean body mass. Extensive loss of lean body mass is often triggered by an inflammatory process and is usually associated with increased plasma levels of cytokines (e.g. TNF-α), which increase the production of α-MSH in the brain. Activation of MC4 receptors in the hypothalamus by α-MSH reduces appetite and increases energy expenditure.

Experimental evidence in tumor bearing mice suggests that cachexia can be prevented or reversed by genetic MC4 receptor knockout or MC4 receptor blockade. The increased body weight in the treated mice is attributable to a larger amount of lean body mass, which mainly consists of skeletal muscle (Marks D. L. et al. Role of the central melanocortin system in cachexia. Cancer Res. (2001) 61: 1432-1438).

Elevated levels of cytokines (e.g. leptin) are likely to be the cause of uremia-associated cachexia in patients with chronic kidney disease (CKD). It was shown that administration of agouti-related peptide (AgRP), an endogenous melanocortin-4 receptor inverse agonist, ameloriated uremic cachexia in mice with CKD. Gains in total body weight and lean body mass were observed along with increased food intake and lower basal metabolic rate. Furthermore, uremic cachexia in mice having a genetic MC4-R knockout was attenuated (Cheung W. et al. Role of leptin and melanocortin signaling in uremia associated cachexia. J. Clin. Invest. (2005) 115: 1659-1665). Intraperitoneal administration of small molecule MC4-R antagonist NBI-12i to uremic mice resulted in similar findings (Cheung W. et al. Peripheral administration of the melanocortin-4 receptor antagonist NBI-12i ameloriates uremia-associated cachexia in mice. J. Am. Soc. Nephrol. (2007) 18: 2517-2524).

Rats with chronic heart failure (CHF) show an impaired ability to accumulate and maintain fat mass and lean body mass. Treatment of aortic banding induced CHF in rats with AgRP resulted in significantly increases in weight gain, lean body mass, fat accumulation, kidney weights and liver weights. (Batra A. K. et al. Central melanocortin blockage attenuates cardiac cachexia in a rat model of chronic heart failure. American Federation for Medical Research, 2008 Western Regional Meeting, abstract 379).

Radiation therapy in cancer patients has been associated with anorexia and nausea (Van Cutsem E., Arends J. The causes and consequences of cancer-associated malnutrition. Eur. J. Oncol. Nurs. (2005) 9 (Suppl 2): 51-63). In a model of radiation induced anorexia in mice, AgRP treated mice ate significantly more food than animals which underwent whole body radiation (RAD) and were vehicle treated. They showed a significantly reduced loss in weight compared to RAD mice treated with vehicle (Joppa M. A. et al. Central infusion of the melanocortin receptor antagonist agouti-related peptide (AgRP(83-132)) prevents cachexia-related symptoms induced by radiation and colon-26 tumors in mice. Peptides (2007) 28: 636-642)

Clinical observations indicate that progression of amyotrophic lateral sclerosis (ALS) might be inversely correlated with body weight (e.g. Ludolph A. C., Neuromuscul Disord. (2006) 16 (8): 530-8). Accordingly, MC-4R inhibitors could be used to treat ALS patients.

Experimental evidence in rats suggests the involvement of central MC4-R in the mechanism of development of tolerance and dependence following chronic morphine administration. Co-administration of the melanocortin-4 receptor antagonist HS014 during chronic morphine treatment delayed the development of tolerance and prevented withdrawal hyperalgesia (Annasaheb S. K. et al. Central administration of selective melanocortin 4 receptor antagonist HS014 prevents morphine tolerance and withdrawal hyperalgesia. Brain Research (2007) 1181: 10-20).

Melanocortin-4 receptor modulators have been previously described in the literature. For example, substituted phenylpiperidine derivatives have been synthesized and explored as MC-4R agonists as well as antagonists.

Benzimidazoles and imidazo[4,5-b]pyridines are described in WO 2004/075823 and WO 2005/056533 as having a good affinity with certain subtypes of melanocortin receptors, particularly MC4 receptors. The claimed affinity, however, is not substantiated with any data stemming from pharmaceutical tests.

Imidazopyridines are further reported in WO 2006/135667 to act as inhibitors of 11-beta hydroxysteroid dehydrogenase type 1. According to WO 2006/094235, such fused heterocyclic compounds may also be useful as sirtuin modulators. WO 2003/006471 proposes the use of heteroaryl substituted fused bicyclic heteroaryl compounds, including imidazopyridines, as GABAA receptor ligands.

In view of the unresolved deficiencies in treatment of various diseases and disorders as discussed above, it is an object of the present invention to provide novel compounds with improved ability to cross the blood brain barrier, which are useful as melanocortin-4 receptor antagonists to treat cachexia induced by e.g. cancer, chronic kidney disease (CKD) or chronic heart failure (CHF), muscle wasting, anorexia induced by e.g. chemotherapy or radiotherapy, anorexia nervosa, amytrophic lateral sclerosis (ALS), pain, neuropathic pain, anxiety and depression and other diseases with MC-4R involvement.

Surprisingly, it has been found that novel imidazopyridines according to formula (I) shown below solve the object of the present invention.

### SUMMARY OF THE INVENTION

The present invention relates to substituted imidazopyridine derivatives of structural formula (I)

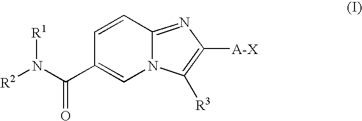

wherein R¹, R², R³, A and X are defined as described below.

The imidazopyridine derivatives of structural formula (I) are effective as melanocortin receptor modulators and are particularly effective as selective melanocortin-4 receptor (MC-4R) antagonists. They are therefore useful for the treatment of disorders where the inactivation of the MC-4R is involved. The antagonists are useful for the treatment of disorders and diseases such as cachexia induced by e.g. cancer, chronic kidney disease (CKD) or chronic heart failure (CHF), muscle wasting, anorexia induced by e.g. chemotherapy or radiotherapy, anorexia nervosa, amyotrophic lateral sclerosis (ALS), pain, neuropathic pain, anxiety and depression.

The present invention also relates to pharmaceutical compositions comprising the compounds of the present invention and a pharmaceutically acceptable carrier.

### DETAILED DESCRIPTION OF THE INVENTION

The present invention relates to substituted imidazopyridine derivatives useful as melanocortin receptor modulators, in particular, selective MC-4R antagonists.

Substituted N-benzyl-N-methyl-2-phenyl-5-diethylamido-3-methylamino-imidazo[1,2-a]pyridines are known from WO-A-02/066478 which describes antagonists of gonadotropin releasing hormone. The present invention relates to novel imidazopyridines which are used as antagonists of MC-4R.

The compounds of the present invention are represented by structural formula (I)

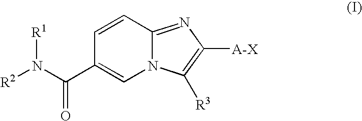

and enantiomers, diastereomers, tautomers, solvates and pharmaceutically acceptable salts thereof,
wherein
R¹ and R² are independently from each other selected from
   H,
   C₁₋₆ alkyl,
   C₁₋₆ alkylene-O—C₁₋₆alkyl
   C₁₋₃ alkylene-heterocyclyl, and
   C₁₋₆ alkylene-C₃₋₇cycloalkyl, or
R¹ and R², together with the nitrogen atom to which they are attached to, form a 5 to 6-membered ring which may additionally contain one oxygen atom in the ring and which is unsubstituted or substituted by one or more substituents selected from OH, C₁₋₆alkyl, O—C₁₋₆alkyl, C₀₋₃alkylene-C₃₋₅cycloalkyl, C₁₋₆alkyl-O—C₁₋₆alkyl and (CH₂)₀₋₃-phenyl;
A is
   —NH—,
   —C₁₋₆alkylene,
   —C₂₋₆alkenylene,
   —C₂₋₆alkinylene or a bond
   wherein alkylene, alkenylene and alkinylene are unsubstituted or substituted with one or more R⁷;
R⁷ is independently selected from
   C₁₋₆alkyl,
   OR¹⁴,
   NR^{15a}R^{15b},
   halogen,
   phenyl and
   heteroaryl,
   wherein phenyl and heteroaryl are unsubstituted or substituted by 1 to 3 R^{4a};
X is
   CN,
   C₃₋₈cycloalkyl, unsubstituted or substituted with one or more halogen atoms, 4- to 8-membered saturated or unsaturated heterocyclyl containing 3 or 4 heteroatoms independently selected from N, O and S,
   5- to 6-membered heteroaryl containing 3 or 4 heteroatoms independently selected from N, O and S,
   5- to 6-membered heteroaryl containing 1 to 3 heteroatoms independently selected from N, O and S, where the heteroaryl ring is fused with a 4- to 8-membered saturated or unsaturated heterocyclyl containing 1 to 3 heteroatoms independently selected from N, O and S, or fused with a 5- to 6-membered heteroaryl containing 1 to 3 heteroatoms independently selected from N, O and S,
   —C(O)—R⁶,
   —OR¹⁴,
   halogen or
   NR^{15a}R^{15b},
   wherein each heterocyclyl or heteroaryl is unsubstituted or substituted by 1 to 3 R^{4a} and/or 1 R^{4b} and/or 1 R⁵;
R^{4a} is
   halogen,
   CN,
   C₁₋₆alkyl, unsubstituted or substituted with one or more substituents selected from halogen atoms, C₁₋₆alkyl, O—C₁₋₆alkyl and OH,
   O—C₁₋₆alkyl, wherein alkyl is unsubstituted or substituted with one or more substituents selected from halogen atoms and OH,
   C₃₋₈cycloalkyl, unsubstituted or substituted with one or more substituents selected from halogen atoms and OH, or
   OH;
R^{4b} is
   C(O)NH₂,
   C(O)NH—C₁₋₆alkyl,
   C(O)N—(C₁₋₆alkyl)₂,
   SO₂—C₁₋₆alkyl,
   C(O)NH—SO₂—C₁₋₆alkyl,
   oxo, whereby the ring is at least partially saturated,
   NH₂,
   NH—C₁₋₆alkyl,
   N—(C₁₋₆alkyl)₂,
   NH—SO₂—CH₃, or NH—SO₂—CF₃;
R⁵ is 5 to 6-membered saturated or unsaturated heterocyclyl containing 1 to 3 heteroatoms independently selected from N, O and S
wherein each heterocyclyl and heteroaryl is unsubstituted or substituted by 1 or 2 R^{4a};
R⁶ is
   OH,
   O—C₁₋₆alkyl, wherein alkyl is unsubstituted or substituted with one or more R¹⁶,
   4- to 8-membered heterocyclyl containing 1 to 3 heteroatoms independently selected from N, O and S, or
      NR^{16a}R^{16b}
   wherein heterocyclyl is unsubstituted or substituted by 1 or 2 R^{4a};
R³ is —(CR⁸R⁹)ₙ-T;
R⁸ and R⁹ are independently from each other selected from
   H,
   OH,
   halogen,
   C₁₋₆alkyl, and
   O—C₁₋₆alkyl,
n is 1 to 6;
T is

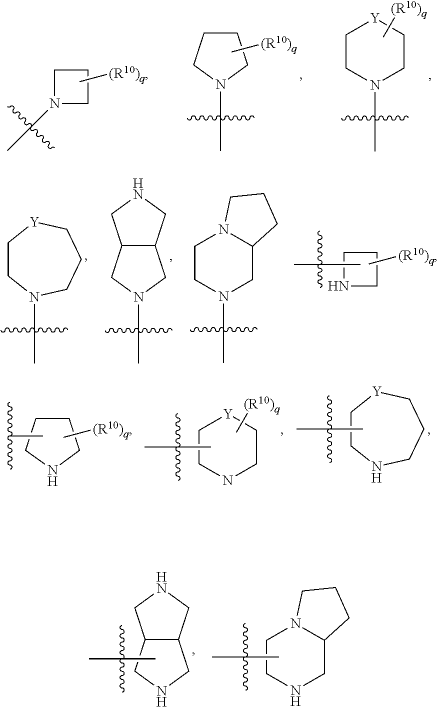

or NR¹²R¹³;
R¹⁰ is
   H,
   OH,
   NH₂,
   C₁₋₆alkyl,
   halogen,
   NH(C₁₋₆alkyl),
   N(C₁₋₆alkyl)₂,
   phenyl or
   heteroaryl,
   wherein phenyl and heteroaryl are unsubstituted or substituted by 1 to 3 R^{4a};
q is 1 or 2;
Y is
   CH₂,
   NR¹¹ or
   O;
R¹¹ is
   H,
   C₁₋₆alkyl or
   (CH₂)₀₋₆—C₃₋₇cycloalkyl;
R¹² and R¹³ are independently from each other selected from
   H,
   C₁₋₆ alkyl,
   (CH₂)₀₋₂—C₃₋₇cycloalkyl and
   C₁₋₆alkylene-O—C₁₋₆alkyl;
   wherein alkyl, alkylene and cycloalkyl are unsubstituted or substituted by 1 to 3 R^{4a};
R¹⁴ is
   H,
   C₁₋₆alkyl, unsubstituted or substituted with one or more substituents selected from halogen,
   phenyl or
   heteroaryl,
   wherein phenyl and heteroaryl are unsubstituted or substituted by 1 to 3 R^{4a};
R^{15a} and R^{15b} are independently from each other selected from
   H,
   C₁₋₆alkyl, unsubstituted or substituted with one or more substituents selected from halogen, OH, O(C₁₋₆alkyl), NH₂, NH(C₁₋₆alkyl) and N(C₁₋₆alkyl)₂,
   C(O)C₁₋₆alkyl,
   C(O)OC₁₋₆alkyl,
   phenyl,
   heteroaryl and
   phenyl fused with a 5- to 6-membered saturated or unsaturated heterocyclyl containing 1 to 3 heteroatoms independently selected from N, O and S, or fused with a 5- to 6-membered heteroaryl containing 1 to 3 heteroatoms independently selected from N, O and S,
   wherein phenyl and heteroaryl are unsubstituted or substituted by 1 to 3 R^{4a};
R¹⁶, R^{16a} and R^{16b} are independently from each other selected from
   H,
   C₁₋₆alkyl, unsubstituted or substituted with one or more substituents selected from halogen, OH, O(C₁₋₆alkyl), NH₂, NH(C₁₋₆alkyl) and N(C₁₋₆alkyl)₂,
   C₀₋₃alkylene-C₃₋₅cycloalkyl,
   phenyl and
   heteroaryl,
   wherein phenyl and heteroaryl are unsubstituted or substituted by 1 to 3 R^{4a}.

In a preferred embodiment, the variants in compounds of formula (I) have the following meaning:
R¹ and R² are independently from each other selected from
   H,
   C₁₋₆ alkyl,
   C₁₋₆ alkylene-O—C₁₋₆alkyl
   C₁₋₃ alkylene-heterocyclyl, and
   C₁₋₆ alkylene-C₃₋₇cycloalkyl, or
R¹ and R², together with the nitrogen atom to which they are attached to, form a 5 to 6-membered ring which may additionally contain one oxygen atom in the ring and which is unsubstituted or substituted by one or more substituents selected from OH, C₁₋₆alkyl, O—C₁₋₆alkyl, C₀₋₃alkylene-C₃₋₅cycloalkyl, C₁₋₆alkyl-O—C₁₋₆alkyl and (CH₂)₀₋₃-phenyl;
A is
   —NH—,
   —C₁₋₆alkylene,
   —C₂₋₆alkenylene,
   —C₂₋₆alkinylene or
   a bond
   wherein alkylene, alkenylene and alkinylene are unsubstituted or substituted with one or more R⁷;
R⁷ is independently selected from
   C₁₋₆alkyl,
   OR¹⁴,
   NR^{15a}R^{15b},
   NR
   halogen,
   phenyl and
   heteroaryl,
   wherein phenyl and heteroaryl are unsubstituted or substituted by 1 to 3 R^{4a};
X is
   CN,
   C₃₋₈cycloalkyl, unsubstituted or substituted with one or more halogen atoms,
   4 to 8-membered saturated or unsaturated heterocyclyl containing 3 or 4 heteroatoms independently selected from N, O and S,
   5- to 6-membered heteroaryl containing 3 or 4 heteroatoms independently selected from N, O and S,
   —C(O)—R⁶,
   —OR¹⁴,
   halogen or
   NR^{15a}R^{15b},
   wherein each heterocyclyl or heteroaryl is unsubstituted or substituted by 1 to 3 R^{4a} and/or 1 R^{4b} and/or 1 R⁵;
R^{4a} is halogen,
   CN,
   C₁₋₆alkyl, unsubstituted or substituted with one or more halogen atoms,
   O—C₁₋₆alkyl, wherein alkyl is unsubstituted or substituted with one or more halogen atoms, or
   OH;
R^{4b} is
   C(O)NH₂,
   C(O)NH—C₁₋₆alkyl,
   C(O)N—(C₁₋₆alkyl)₂,
   C(O)NH—SO₂—C₁₋₆alkyl,
   oxo, whereby the ring is at least partially saturated,
   NH₂,
   NH—C₁₋₆alkyl,
   N—(C₁₋₆alkyl)₂,
   NH—SO₂—CH₃, or
   NH—SO₂—CF₃;
R⁵ is 5 to 6-membered saturated or unsaturated heterocyclyl containing 1 to 3 heteroatoms independently selected from N, O and S
wherein heterocyclyl is unsubstituted or substituted by 1 or 2 R^{4a};
R⁶ is
   OH,
   O—C₁₋₆alkyl, wherein alkyl is unsubstituted or substituted with one or more R¹⁶, or
   NR^{16a}R^{16b};
R³ is —(CR³R⁹)ₙ-T;
R⁸ and R⁹ are independently from each other selected from
   H,
   OH,
   halogen,
   C₁₋₆alkyl, and
   O—C₁₋₆alkyl,
n is 1 to 6;
T is

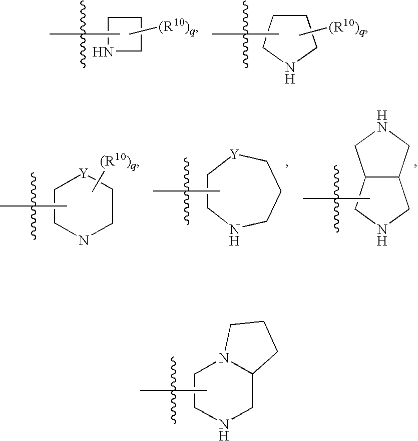

or NR¹²R¹³;
R¹⁰ is
   H,
   NH₂,
   OH,
   C₁₋₆alkyl,
   halogen,
   NH(C₁₋₆alkyl),
   N(C₁₋₆alkyl)₂,
   phenyl or
   heteroaryl,
   wherein phenyl and heteroaryl are unsubstituted or substituted by 1 to 3 R^{4a};
q is 1 or 2;
Y is
   CH₂,
   NR¹¹ or
   O;
R¹¹ is
   H,
   C₁₋₆alkyl or
   (CH₂)₀₋₆—C₃₋₇cycloalkyl;
R¹² and R¹³ are independently from each other selected from
   H,
   C₁₋₆ alkyl,
   (CH₂)₀₋₂—C₃₋₇cycloalkyl and
   C₁₋₆alkylene-O—C₁₋₆alkyl;
   wherein alkyl, alkylene and cycloalkyl are unsubstituted or substituted by 1 to 3 R^{4a};
R¹⁴ is
   H,
   C₁₋₆alkyl, unsubstituted or substituted with one or more substituents selected from halogen,
   phenyl or
   heteroaryl,
   wherein phenyl and heteroaryl are unsubstituted or substituted by 1 to 3 R^{4a};
R^{15a} and R^{15b} are independently from each other selected from
   H,
   C₁₋₆alkyl, unsubstituted or substituted with one or more substituents selected from halogen, OH, O(C₁₋₆alkyl), NH₂, NH(C₁₋₆alkyl) and N(C₁₋₆alkyl)₂,
   phenyl and
   heteroaryl,
   wherein phenyl and heteroaryl are unsubstituted or substituted by 1 to 3 R^{4a}, and
   C(O)C₁₋₆alkyl;
R¹⁶, R^{16a} and R^{16b} are independently from each other selected from
   H,
   C₁₋₆alkyl, unsubstituted or substituted with one or more substituents selected from halogen, OH, O(C₁₋₆alkyl), NH₂, NH(C₁₋₆alkyl) and N(C₁₋₆alkyl)₂,
   C₀₋₃alkylene-C₃₋₅cycloalkyl,
   phenyl and
   heteroaryl,
   wherein phenyl and heteroaryl are unsubstituted or substituted by 1 to 3 R^{4a}.

Further, in a preferred embodiment, the variant A represents —NH— or a bond. More preferably, A represents a bond.

In an equally preferred embodiment, the variant A represents —C₁₋₆alkylene, —C₂₋₆ alkenylene or —C₂₋₆alkinylene wherein alkylene, alkenylene and alkinylene are unsubstituted or substituted with one or more R⁷ such as 1, 2 or 3 R⁷. Preferably, A represents C₁₋₃alkylene, such as methyl, ethyl, propyl or isopropyl, C₂₋₃alkenylene, such as ethenylene or prop-1-enylene, or C₂₋₃alkinylene, such as ethinylene or prop-2-inylene. Most preferably, A represents C₁₋₃alkylene. It is further preferred that alkylene, alkenylene and alkinylene are unsubstituted or substituted by 1 R⁷. More preferably, alkylene, alkenylene and alkinylene are unsubstituted.

R⁷ is as defined above. Preferably, R⁷ represents C₁₋₆alkyl, OR¹⁴, NR^{15a}R^{15b} or halogen, wherein R¹⁴, R^{15a} and R^{15b} are defined as above. More preferably, R⁷ represents C₁₋₆alkyl, OH, NH₂ or fluorine.

It is further preferred that R¹ and R² independently from each other represent C₃₋₆alkyl or that R¹ and R², together with the nitrogen atom to which they are attached to, form a 5 to 6-membered ring which may additionally contain one oxygen atom in the ring and which is unsubstituted or substituted by one or more substituents, preferably 1, 2 or 3 substituents, independently selected from OH, C₁₋₆alkyl, C₀₋₃alkylene-C₃₋₅cycloalkyl, O—C₁₋₆alkyl, C₁₋₆alkylene-O—C₁₋₆alkyl and (CH₂)₀₋₃-phenyl.

More preferably, R¹ and R² independently from each other represent C₃₋₆alkyl.

In a preferred embodiment, the variant T is NR¹²R¹³. Therein, the variants R¹² and R¹³ are preferably independently from each other selected from H, C₁₋₃alkyl and (CH₂)₀₋₂—C₃₋₆ cycloalkyl, wherein alkyl and cycloalkyl are optionally substituted by 1 to 3 R^{4a} such as 1, 2 or 3 substituents R^{4a}.

In an alternative preferred embodiment, the variant T is selected from

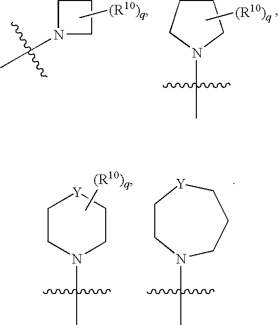

It is preferred that the variant Y is CH₂ or NR¹¹. Preferably, R¹¹ is hydrogen.

It is further preferred that R¹⁰ is selected from H, NH₂, C₁₋₆alkyl, NH(C₁₋₆alkyl) and N(C₁₋₆alkyl)₂. More preferably, R¹⁰ is H, NH₂ or C₁₋₆alkyl.

Regarding the variant X, said variant preferably represents a 4 to 8-membered saturated or unsaturated heterocyclyl containing 3 or 4 heteroatoms independently selected from N, O and S, or a 5- to 6-membered heteroaryl containing 3 or 4 heteroatoms independently selected from N, O and S, wherein each heterocyclyl or heteroaryl is unsubstituted or substituted by 1 to 3 R^{4a} such as 1, 2 or 3 R^{4a} and/or 1 R^{4b} and/or 1 R⁵.

More preferably, X represents

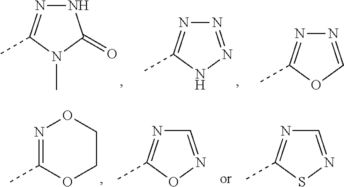

Alternatively, it is preferred that the variant X represents CN, C₃₋₈cycloalkyl, unsubstituted or substituted with one or more halogen atoms, —C(O)—R⁶, OR¹⁴, halogen or NR^{15a}R^{15b}.

In an equally preferred embodiment, the variant X represents —C(O)—R⁶, OR¹⁴ or NR^{16a}R^{15b}. More preferably, X is —C(O)—R⁶ or NR^{15a}R^{15b} most preferably X is —C(O)—R⁶.

R⁶ is preferably O—C₁₋₆alkyl, wherein alkyl is unsubstituted or substituted with one or more R¹⁶.

In an equally preferred embodiment, the variant R⁶ is NR^{16a}R^{16b}, more preferably NH—C₁₋₆ alkyl.

In a further preferred embodiment, the variant R⁶ is a 4- to 8-membered heterocyclyl containing 1 to 3 heteroatoms independently selected from N, O and S.

The index n represents an integer from 1 to 6, such as the integers 1, 2, 3, 4, 5 or 6. Preferably, n represents 1, 2, 3, or 4.

Compounds of the formula (I) in which some or all of the above-mentioned groups have the preferred or more preferred meanings are also an object of the present invention.

In the above and the following, the employed terms have the meaning as described below:

Alkyl is a straight chain or branched alkyl having 1, 2, 3, 4, 5 or 6 carbon atoms, such as methyl, ethyl, n-propyl, isopropyl, n-butyl, isobutyl, tert-butyl, n-pentyl, isopentyl, neopentyl, or hexyl.

Alkenyl is a straight chain or branched alkyl having 1, 2, 3, 4, 5, or 6 carbon atoms and one to three double bonds, preferably one or two double bonds, most preferably one double bound. Preferred examples of a C₂₋₆alkenyl group are ethenyl, prop-1-enyl, prop-2-enyl, isoprop-1-enyl, n-but-1-enyl, n-but-2-enyl, n-but-3-enyl, isobut-1-enyl, isobut-2-enyl, n-pent-1-enyl, n-pent-2-enyl, n-pent-3-enyl, n-pent-4-enyl, n-pent-1,3-enyl, isopent-1-enyl, isopent-2-enyl, neopent-1-enyl, n-hex-1-enyl, n-hex-2-enyl, n-hex-3-enyl, n-hex-4-enyl, n-hex-5-enyl, n-hex-1,3-enyl, n-hex-2,4-enyl, n-hex-3,5-enyl, and n-hex-1,3,5-enyl. More preferred examples of a C₂₋₆alkenyl group are ethenyl and prop-1-enyl.

Alkinyl is a straight chain or branched alkyl having 1, 2, 3, 4, 5, or 6 carbon atoms and one to three triple bonds, preferably one or two triple bonds, most preferably one triple bond.

Preferred examples of a C₂₋₆alkinyl group are ethinyl, prop-1-inyl, prop-2-inyl, n-but-1-inyl, n-but-2-inyl, n-but-3-inyl, n-pent-1-inyl, n-pent-2-inyl, n-pent-3-inyl, n-pent-4-inyl, n-pent-1,3-inyl, isopent-1-inyl, neopent-1-inyl, n-hex-1-inyl, n-hex-2-inyl, n-hex-3-inyl, n-hex-4-inyl, n-hex-5-inyl, n-hex-1,3-inyl, n-hex-2,4-inyl, n-hex-3,5-inyl and n-hex-1,3,5-inyl. More preferred examples of a C₂₋₆alkinyl group are ethinyl and prop-1-inyl.

Cycloalkyl is an alkyl ring having preferably 3, 4, 5, 6, 7 or 8 carbon atoms at the most, such as cyclopropyl, cyclobutyl, cyclopentyl, cyclohexyl, cycloheptyl or cyclooctyl, more preferably 3, 4, 5 or 6 carbon atoms.

Heteroaryl is an aromatic moiety having 1, 2, 3, 4 or 5 carbon atoms and at least one heteratom independently selected from O, N and/or S. Heteroaryl is preferably selected from thienyl, pyrrolyl, imidazolyl, pyrazolyl, pyridyl, pyrazinyl, pyrimidinyl, pyridazinyl, isothiazolyl, isoxazyl, furanyl, and indazolyl, more preferably from thienyl, furanyl, imidazolyl, pyridyl, and pyrimidinyl.

Heterocyclyl is a saturated or unsaturated ring containing at least one heteroatom independently selected from O, N and/or S and 1, 2, 3, 4, 5, 6 or 7 carbon atoms. Preferably, heterocyclyl is a 4, 5, 6, 7 or 8-membered ring and is preferably selected from tetrahydrofuranyl, azetidinyl, pyrrolidinyl, piperidinyl, pyranyl, morpholinyl, thiomorpholinyl, more preferably from piperidinyl and pyrrolidinyl.

Halogen is a halogen atom selected from F, Cl, Br and I, preferably from F, Cl and Br.

The compounds of structural formula (I) are effective as melanocortin receptor modulators and are particularly effective as selective modulators of MC-4R. They are useful for the treatment and/or prevention of disorders responsive to the inactivation of MC-4R, such as cachexia induced by e.g. cancer, chronic kidney disease (CKD) or chronic heart failure (CHF), muscle wasting, anorexia induced by e.g. chemotherapy or radiotherapy, anorexia nervosa, amyotrophic lateral sclerosis (ALS), pain, neuropathic pain, anxiety and depression and other diseases with MC-4R involvement.

### Optical Isomers—Diastereomers—Geometric Isomers—Tautomers

Compounds of structural formula (I) contain one or more asymmetric centers and can occur as racemates and racemic mixtures, single enantiomers, diastereomeric mixtures and individual diastereomers. The present invention is meant to comprehend all such isomeric forms of the compounds of structural formula (I).

Compounds of structural formula (I) may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general formula (I) may be obtained by stereospecific synthesis using optically pure starting materials or reagents of known absolute configuration.

### Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, lithium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine, caffeine, choline, N,N′-dibenzylethylenediamine, diethylamine, 2-diethylaminoethanol, 2-dimethylamino-ethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, formic, furnaric, gluconic, glutamic, hydrobromic, hydrochloric, isethionic, lactic, maleic, malic, mandelic, methanesulfonic, malonic, mucic, nitric, parnoic, pantothenic, phosphoric, propionic, succinic, sulfuric, tartaric, p-toluenesulfonic, trifluoroacetic acid and the like. Particularly preferred are citric, fumaric, hydrobromic, hydrochloric, maleic, phosphoric, sulfuric and tartaric acids.

It will be understood that, as used herein, references to the compounds of formula (I) are meant to also include the pharmaceutically acceptable salts.

### Utility

Compounds of formula (I) are melanocortin receptor antagonists and as such are useful in the treatment, control or prevention of diseases, disorders or conditions responsive to the inactivation of one or more of the melanocortin receptors including, but not limited to, MC-1R, MC-2R, MC-3R, MC-4R or MC-5R. Such diseases, disorders or conditions include, but are not limited to, cachexia induced by e.g. cancer, chronic kidney disease (CKD) or chronic heart failure (CHF), muscle wasting, anorexia induced by e.g. chemotherapy or radiotherapy, anorexia nervosa, amyotrophic lateral sclerosis (ALS), pain, neuropathic pain, anxiety and depression.

The compounds of formula (I) can be further used in the treatment, control or prevention of diseases, disorders or conditions which are responsive to the inactivation of one or more melanocortin receptors including, but not limited to, MC-1R, MC-2R, MC-3R, MC-4R or MC-5R. Such diseases, disorders or conditions include, but are not limited to, hypertension, hyperlipidemia, osteoarthritis, cancer, gall bladder disease, sleep apnea, compulsion, neuroses, insomnia/sleep disorder, substance abuse, pain, fever, inflammation, immune-modulation, rheumatoid arthritis, skin tanning, acne and other skin disorders, neuroprotective and cognitive and memory enhancement including the treatment of Alzheimer's disease.

### Administration and Dose Ranges

Any suitable route of administration may be employed for providing a mammal, especially a human with an effective dosage of a compound of the present invention. For example, oral, rectal, topical, parenteral, ocular, pulmonary, nasal and the like may be employed. Dosage forms include tablets, troches, dispersions, suspensions, solutions, capsules, creams, ointments, aerosols and the like. Preferably compounds of formula (I) are administered orally or topically.

The effective dosage of active ingredient employed may vary depending on the particular compound employed, the mode of administration, the condition being treated and the severity of the condition being treated. Such dosage may be ascertained readily by a person skilled in the art.

When treating cachexia induced by e.g. cancer, chronic kidney disease (CKD) or chronic heart failure (CHF), muscle wasting, anorexia induced by e.g. chemotherapy or radiotherapy, anorexia nervosa, amyotrophic lateral sclerosis (ALS), pain, neuropathic pain, anxiety and depression generally satisfactory results are obtained when the compounds of the present invention are administered at a daily dosage of from about 0.001 milligram to about 100 milligrams per kilogram of body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

### Formulation

The compounds of formula (I) are preferably formulated into a dosage form prior to administration. Accordingly the present invention also includes a pharmaceutical composition comprising a compound of formula (I) and a suitable pharmaceutical carrier.

The present pharmaceutical compositions are prepared by known procedures using well-known and readily available ingredients. In making the formulations of the present invention, the active ingredient (a compound of formula (I)) is usually mixed with a carrier, or diluted by a carrier, or enclosed within a carrier, which may be in the form of a capsule, sachet, paper or other container. When the carrier serves as a diluent, it may be a solid, semisolid or liquid material which acts as a vehicle, excipient or medium for the active ingredient. Thus, the compositions can be in the form of tablets, pills, powders, lozenges, sachets, cachets, elixirs, suspensions, emulsions, solutions, syrups, aerosol (as a solid or in a liquid medium), soft and hard gelatin capsules, suppositories, sterile injectable solutions and sterile packaged powders.

Some examples of suitable carriers, excipients and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl and propylhydroxybenzoates, talc, magnesium stearate and mineral oil. The formulations can additionally include lubricating agents, wetting agents, emulsifying and suspending agents, preserving agents, sweetening agents or flavoring agents. The compositions of the invention may be formulated so as to provide quick, sustained or delayed release of the active ingredient after administration to the patient.

### Preparation of Compounds of the Invention

The compounds of formula (I) when existing as a diastereomeric mixture, may be separated into diastereomeric pairs of enantiomers by fractional crystallization from a suitable solvent such as methanol, ethyl acetate or a mixture thereof. The pair of enantiomers thus obtained may be separated into individual stereoisomers by conventional means by using an optically active acid as a resolving agent. Alternatively, any enantiomer of a compound of the formula (I) may be obtained by stereospecific synthesis using optically pure starting materials or reagents of known configuration.

The compounds of formula (I) of the present invention can be prepared according to the procedures of the following Schemes and Examples, using appropriate materials and are further exemplified by the following specific examples. Moreover, by utilizing the procedures described herein, in conjunction with ordinary skills in the art, additional compounds of the present invention claimed herein can be readily prepared. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. The instant compounds are generally isolated in the form of their pharmaceutically acceptable salts, such as those described previously. The free amine bases corresponding to the isolated salts can be generated by neutralization with a suitable base, such as aqueous sodium hydrogencarbonate, sodium carbonate, sodium hydroxide and potassium hydroxide, and extraction of the liberated amine free base into an organic solvent followed by evaporation. The amine free base isolated in this manner can be further converted into another pharmaceutically acceptable salt by dissolution in an organic solvent followed by addition of the appropriate acid and subsequent evaporation, precipitation or crystallization. All temperatures are degrees Celsius.

In the schemes, preparations and examples below, various reagent symbols and abbreviations have the following meanings
AcOH acetic acid
Ac₂O acetic anhydride
Boc tert-butoxycarbonyl
CDI 1,1′-carbonyldiimidazole
DCE 1,2-dichloroethane
DCM dichloromethane
DIBAL-H diisobutylaluminiumhydride
DIEA ethyl-diisopropylamine
DMF N,N-dimethylformamide
DMSO dimethylsulfoxide
EDC 1-(3-dimethylaminopropyl)-3-ethylcarbodiimide hydrochloride
EtOAc ethyl acetate
HOBt 1-hydroxybenzotriazole hydrate
h hour(s)
iBu isobutyl
MeCN acetonitrile
MeLi methyllithium
MeOH methanol
Ms mesyl
NIS N-iodosuccinimide
NMM N-methylmorpholine
MW molecular weight
NBS N-bromosuccinimide
NIS N-iodosuccinimide
PdCl₂(PPh₃)₂ bis(triphenylphosphine)palladium(II) dichloride
Pd₂ dba₃ tris(dibenzylideneacetone)dipalladium(0)
Pd(dppf)₂Cl₂ 1,1′-bis(diphenylphosphino)ferrocene-palladium(II)dichloride dichloromethane adduct
Pd(PPh₃)₄ tetrakis(triphenylphosphine)palladium(0)
RT room temperature
TEA triethylamine
TFAA trifluoroacetic acid anhydride
THF tetrahydrofurane
t_{R} (min) HPLC retention time
T_{S} tosyl
Xantphos 9,9-dimethyl-4,5-bis(di-tert-butylphosphino)xanthene

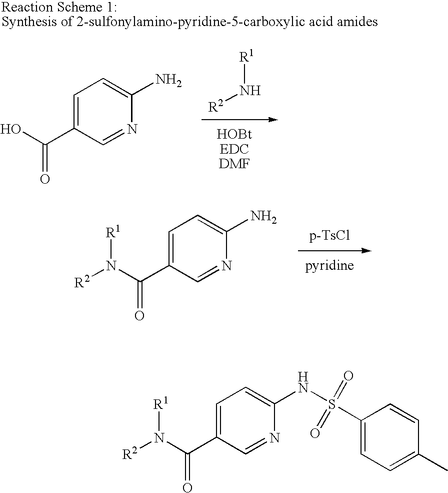

As shown in Reaction Scheme 1, optionally substituted amine and 6-amino-nicotinic acid are reacted in an amide coupling reaction in the presence of a coupling reagent such as EDC in an organic solvent such as DMF or DCM at a suitable temperature. The resulting 6-amino-nicotinic acid amide can then be reacted with a sulfonylchloride in a solvent such as pyridine or any other appropriate solvent and an organic base such as triethylamine to yield the corresponding sulfonylamino-nicotinamides.

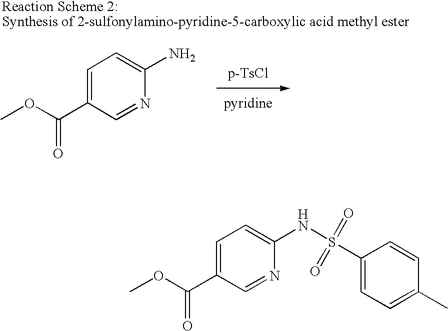

Alternatively, 2-amino-pyridine-5-carboxylic acid methyl ester can be reacted under the conditions described above to yield the corresponding sulfonylamino-esters, as shown in Reaction Scheme 2.

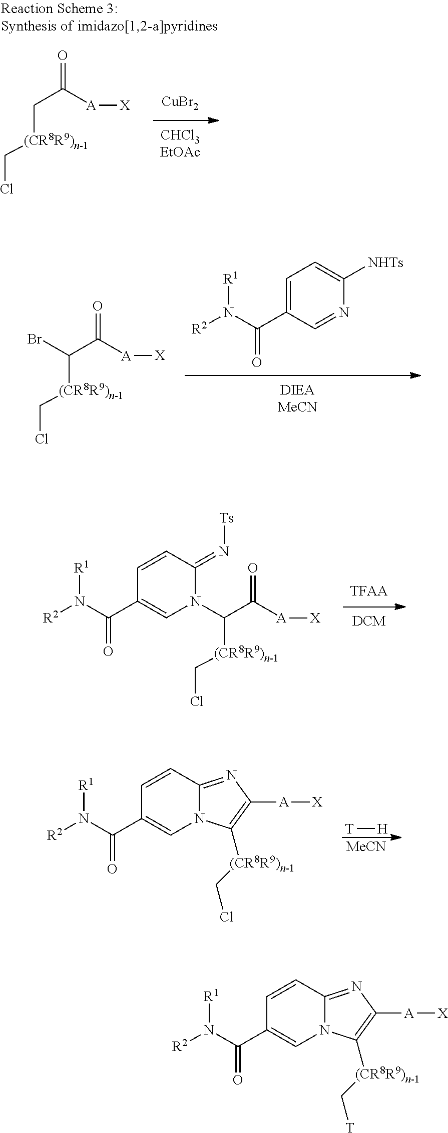

As shown in Reaction Scheme 3, optionally substituted α-bromoketones can be obtained from the corresponding ketone by reacting it for example with copper(II) bromide in a solvent such as a mixture of chloroform and ethyl acetate at an appropriate temperature for a given time. The resulting α-bromoketones can then be reacted with sulfonylamino-nicotinamides in a solvent such as MeCN in the presence of an appropriate base, for example DIEA, to yield the N-alkylated sulfonylamino-nicotinamides. These intermediates can then be further cyclised to the imidazo[1,2-a]pyridines by treating them with TFAA in a suitable solvent such as DCM or 1,2-dichloroethane at an appropriate temperature for a given time. Chloroalkyl-substituted imidazo[1,2-a]pyridines can be reacted with a capping group T-H in a solvent such as acetonitrile at elevated temperature to yield the title compounds. In some cases a base may be added to liberate the free base of T-H.

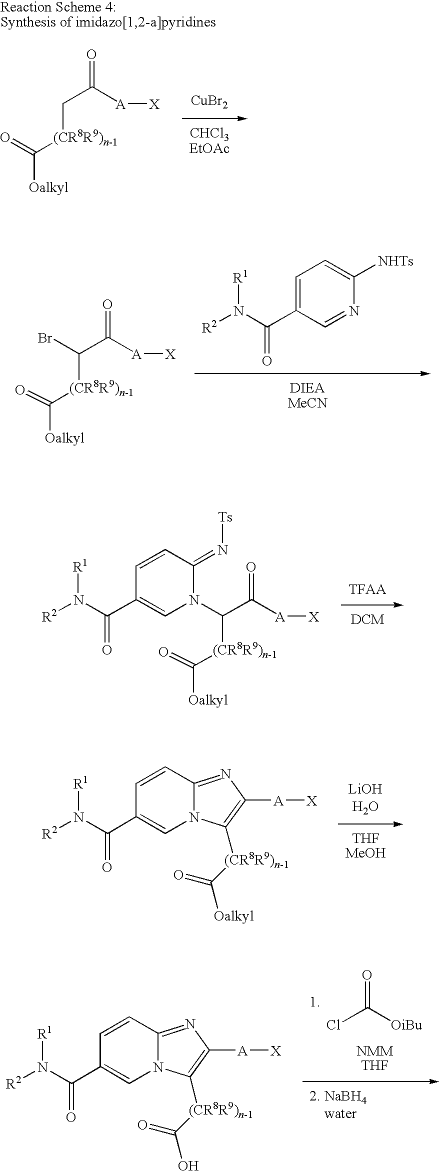

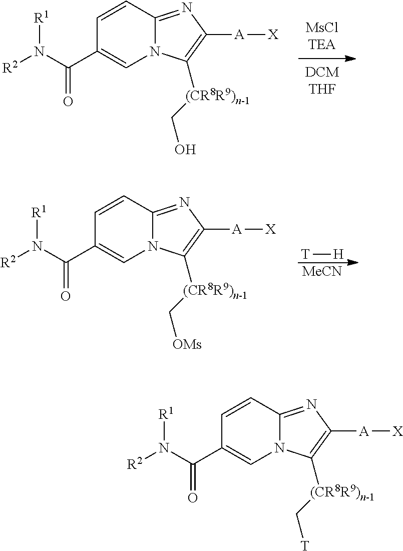

As shown in Reaction Scheme 4, optionally substituted ω-alkoxycarbonyl-α-bromoketones can be obtained from the corresponding ketone by reacting it for example with copper(II) bromide in a solvent such as mixture of ethyl acetate and chloroform at an appropriate temperature for a given time. The resulting α-bromoketones can then be reacted with sulfonylamino-amides in a solvent such as MeCN in the presence of an appropriate base, for example DIEA, to yield the N-alkylated sulfonylamino-amides. These intermediates can then be further cyclised to the corresponding imidazo[1,2-a]pyridines by treating them with TFAA in a suitable solvent such as DCM or 1,2-dichloroethane at an appropriate temperature for a given time. Ester function of optionally substituted imidazo[1,2-a]pyridines can be hydrolyzed under basic conditions using a reagent like lithium hydroxide monohydrate in a suitable solvent such as a mixture of water, THF and MeOH. The resulting acid can be activated with a reagent such as isobutyl chloroformate or CDI in the presence of a suitable base such as N-methylmorpholine in an appropriate solvent such as THF and subsequently be reduced to the corresponding alcohol with a reducing agent such as sodium borohydride in an appropriate solvent such as a mixture of THF and water. The alcohol function can be converted to a leaving group with a reagent such as mesyl chloride or tosyl chloride in an appropriate solvent such as mixture of DCM and THF in the presence of a suitable base like TEA. Product of this reaction can be treated with an amine T-H in an appropriate solvent like MeCN to yield the target molecule.

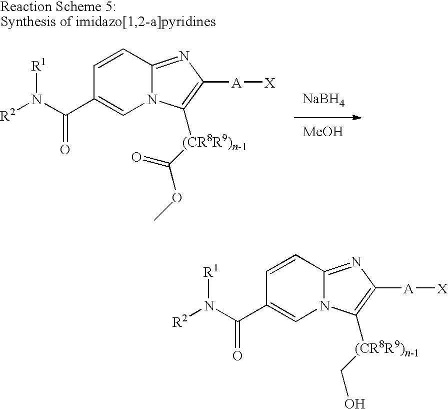

As shown in Reaction Scheme 5, methyl ester functions of optionally substituted imidazo[1,2-a]pyridines can be reduced to the corresponding alcohol with a reagent such as sodium borohydride in an appropriate solvent like methanol. The alcohol can be further reacted to the target molecules as depicted in Reaction Scheme 4.

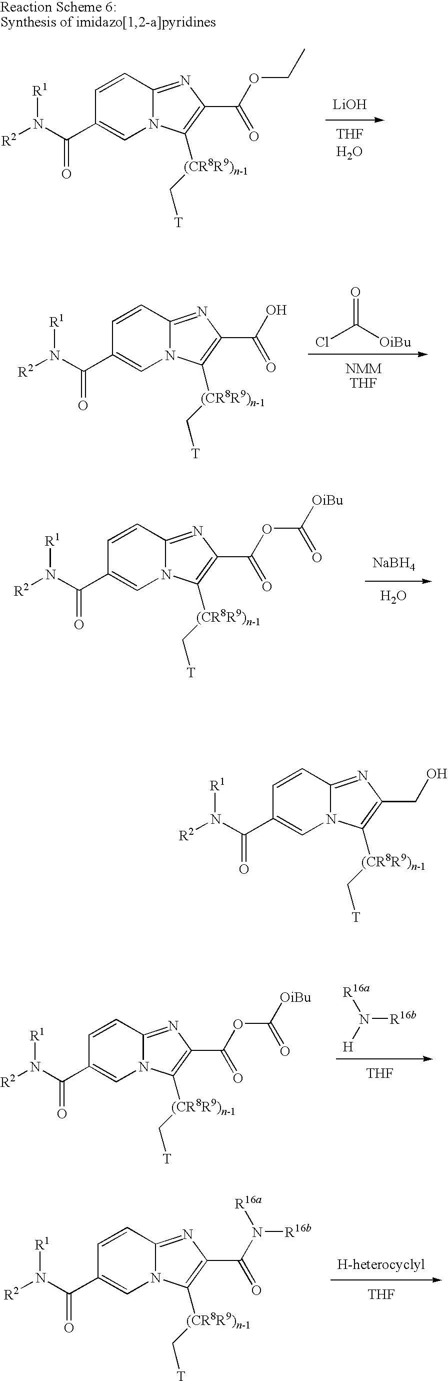

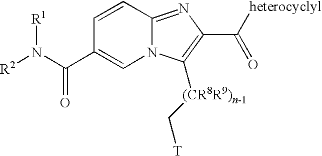

As shown in Reaction Scheme 6, optionally substituted imidazo[1,2-a]pyridine-2-carboxylic acid ethyl ester can be saponified using a base such as lithium hydroxide in a suitable solvent such as a mixture of water and tetrahydrofuran. The corresponding acid can be activated with a reagent such as isobutylchloroformate in an appropriate solvent such as tetrahydrofuran in the presence of a suitable base like N-methylmorpholine at a suitable temperature. The activated species can be reacted with a reducing agent such as sodium borohydride in a solvent such as water to yield the corresponding primary alcohol. Alternatively, optionally substituted mixed anhydride can also be reacted with amines in a suitable solvent such as tetrahydrofuran or a mixture of THF and water to yield the corresponding amides.

Generally after the reaction is completed, the solvent is evaporated and the reaction mixture can be diluted with an appropriate organic solvent, such as EtOAc or DCM, which is then washed with aqueous solutions, such as water, HCl, NaHSO₄, bicarbonate, NaH₂PO₄, phosphate buffer (pH 7), brine, Na₂CO₃ or any combination thereof. The reaction mixture can be concentrated and then be partitioned between an appropriate organic solvent and an aqueous solution. Alternatively, the reaction mixture can be concentrated and subjected to chromatography without aqueous workup.

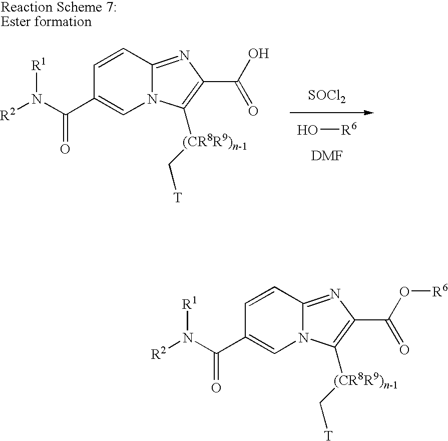

Imidazo[1,2-a]pyridines bearing an alkoxycarbonyl group in 2-position can be obtained as depicted in Reaction Scheme 7. Optionally substituted imidazo[1,2-a]pyridines-2-carboxylic acids or their lithium salts can be reacted with a reagent such as thionyl chloride or oxalyl chloride in an alcohol HO—R⁶ in the presence of a catalyst like DMF to form the corresponding ester.

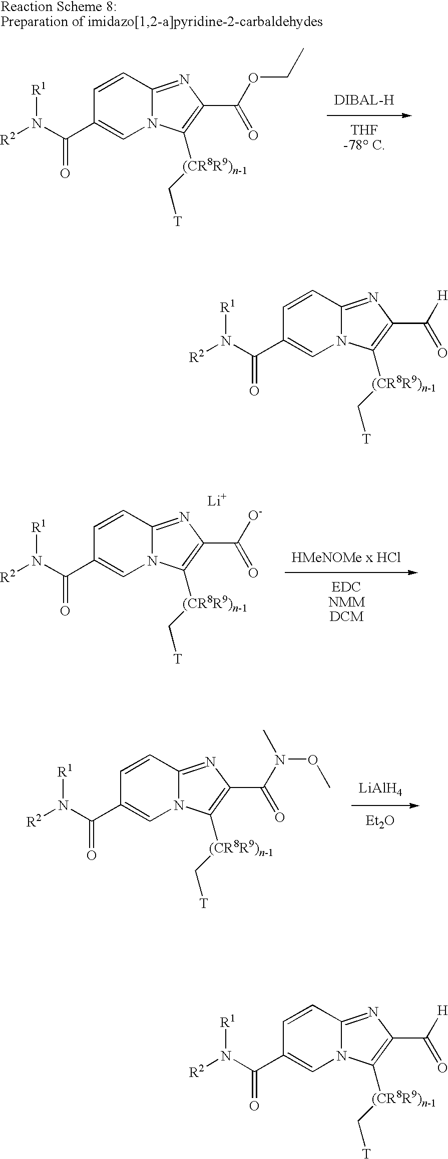

As shown in Reaction Scheme 8, optionally substituted imidazo[1,2-a]pyridine-2-carboxylic acid ethyl ester can be reduced to the corresponding aldehyde using a reagent like DIBAL-H in a suitable solvent such as THF at an appropriate temperature. Alternatively, optionally substituted imidazo[1,2-a]pyridine-2-carboxylic-3-carbaldehydes can also be obtained starting from the corresponding acid or lithium salt of said acid. Reaction with N,O-dimethylhydroxylamine hydrochloride and a coupling reagent like EDC and in the presence of a reagent such as HOBt and a base like NMM in an appropriate solvent such as DCM provides the corresponding Weinreb amide which can subsequently be reduced to the desired aldehyde with a reducing agent such as lithium aluminium hydride in an inert solvent like diethyl ether at an appropriate temperature.

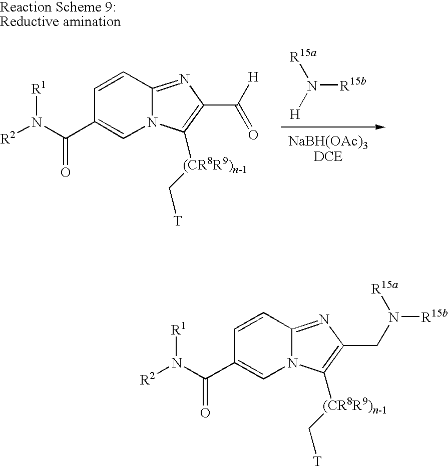

Optionally substituted imidazo[1,2-a]pyridine-2-carboxylic-3-carbaldehydes can be reductively aminated as shown in Reaction Scheme 9. Reaction with an amine HNR^{15a}R^{15b} in the presence of a reagent like sodium triacetoxyborohydride in a solvent such as 1,2-dichloroethane yields the target compounds.

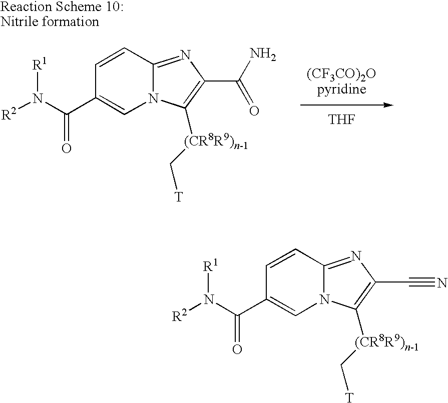

Imidazo[1,2-a]pyridines bearing an nitrile group in 2-position can be obtained as depicted in Reaction Scheme 10. Optionally substituted imidazo[1,2-a]pyridines-2-carboxylic acid amides can be reacted with a reagent such as trifluoroacetic acid anhydride in an appropriate solvent like THF in the presence of a base such as pyridine at a suitable temperature to yield the target compound.

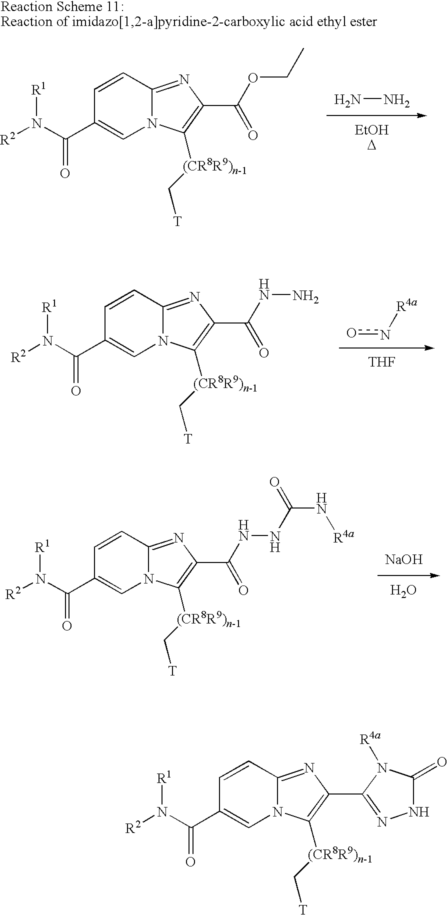

Optionally substituted imidazo[1,2-a]pyridine-2-carboxylic acid ethyl esters can be transformed to 5-imidazo[1,2-a]pyridin-2-yl-2,4-dihydro-[1,2,4]triazol-3-ones as depicted in Reaction Scheme 11. Reaction of the ester with hydrazine monohydrate in a solvent like ethanol at elevated temperature yields the acid hydrazide which can be further reacted with optionally substituted isocyanates O═C═N—R^{4a} in an appropriate solvent such as THF. Cyclization can be achieved by treating the product of this reaction with aqueous sodium hydroxide solution at elevated temperatures e.g. in a microwave reactor.

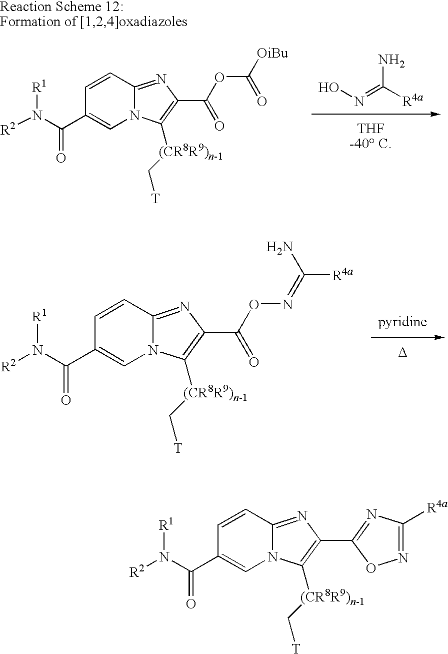

As shown in Reaction Scheme 12, optionally substituted imidazo[1,2-a]pyridine-2-carboxylic acids, activated as described above, can be reacted with N-hydroxyamidines in a suitable solvent such as THF at an appropriate temperature. Cyclization to the [1,2,4] oxadiazoles can be achieved by subsequent heating of the O-acyl amidoxime intermediate in a solvent like pyridine.

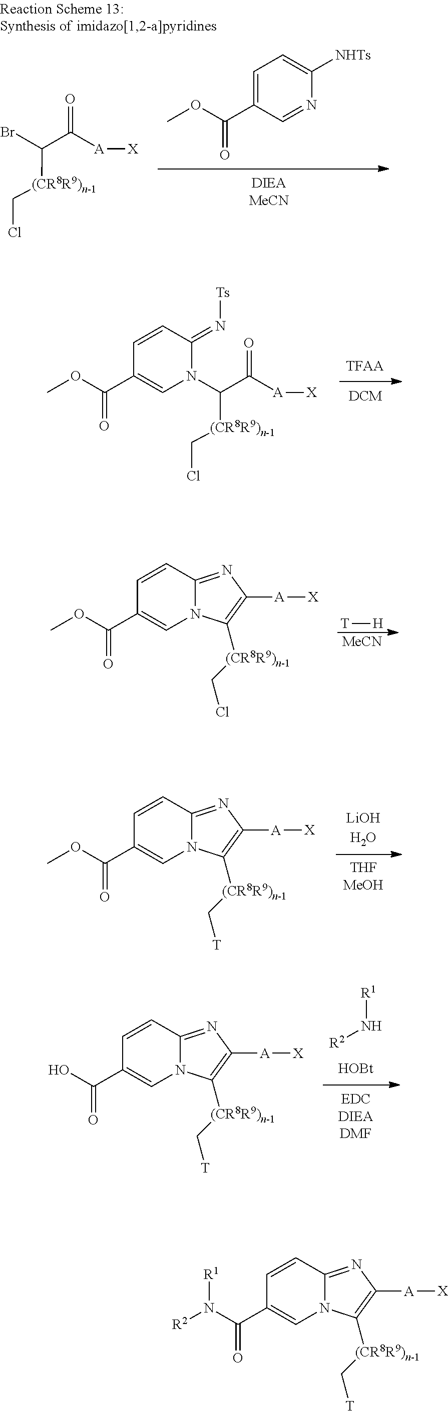

As shown in Reaction Scheme 13, optionally substituted ω-chloro-α-bromoketones can also be reacted with a sulfonylamino-ester in a solvent such as MeCN in the presence of an appropriate base, for example DIEA, to yield the N-alkylated sulfonylamino-esters. These intermediates can then be further cyclised to the corresponding imidazo[1,2-a]pyridines by treating them with TFAA in a suitable solvent such as DCM or 1,2-dichloroethane at an appropriate temperature for a given time. The capping group T can be inserted by reacting the chloroalkyl substituted imidazo[1,2-a]pyridines with a capping group T-H in an appropriate solvent such as MeCN. When T-H is used in form of a hydrochloride, a suitable base such as DIEA is used in addition to liberate the free amine T-H. Ester function of optionally substituted imidazo[1,2-a]pyridines can be hydrolyzed under basic conditions using a reagent like lithium hydroxide monohydrate in a suitable solvent such as a mixture of water, THF and MeOH. The product of the saponification can be isolated as lithium salt or as the corresponding acid. Alternatively, the ester function can also be cleaved under acidic conditions for example using a reagent such as aqueous hydrochloric acid. The product of the ester cleavage can be introduced into the next step as acid or lithium salt. Amide formation can be achieved using standard peptide coupling procedures. The acid can be coupled with an amine HNR¹R² in the presence of EDC/HOBt, EDC/HOAt, HATU, a base such as diisopropylethylamine and a solvent such as dichloromethane. A suitable solvent, such as DCM, DMF, THF or a mixture of the above solvents, can be used for the coupling procedure. A suitable base includes triethylamine (TEA), diisopropylethylamine (DIEA), N-methylmorpholine (NMM), collidine or 2,6-lutidine. A base may not be needed when EDC/HOBt is used.

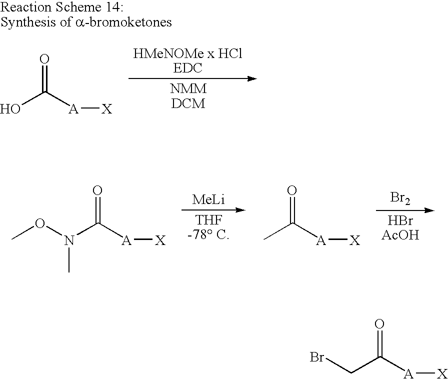

As shown in Reaction Scheme 14 optionally substituted bromoketones can be obtained in a three step reaction sequence starting from carboxylic acids. Said carboxylic acids can be converted to the corresponding Weinreb amides using N,O-dimethylhydroxylamine hydrochloride with a coupling reagent like EDC in the presence of a suitable base like NMM in an appropriate solvent such as DCM. The Weinreb amides can be converted to the corresponding methyl ketones using a reagent such as methyllithium in an inert solvent like THF at a suitable temperature. Bromination can be achieved using a mixture of bromine and hydrogen bromide in acetic acid.

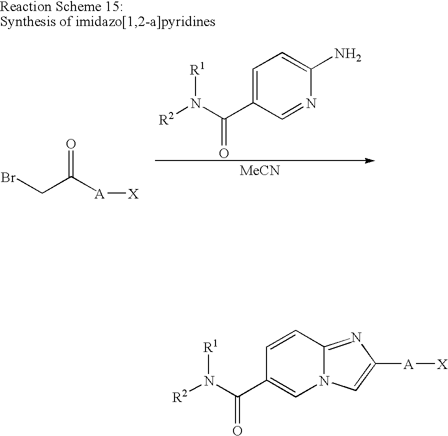

As shown in Reaction Scheme 15 optionally substituted aminopyridine-amides, which can be obtained as shown in Reaction Scheme 1, can be converted to imidazo[1,2-a]pyridine 6-carboxylic acid amides by reaction with α-bromoketones in a solvent like MeCN. This reaction can be carried out either in a flask in refluxing solvent or any other appropriate temperature or in a microwave reaction system. The reaction products can be purified by standard procedures or may precipitate directly from the solution upon cooling and may thus be used in subsequent reactions without further purification.

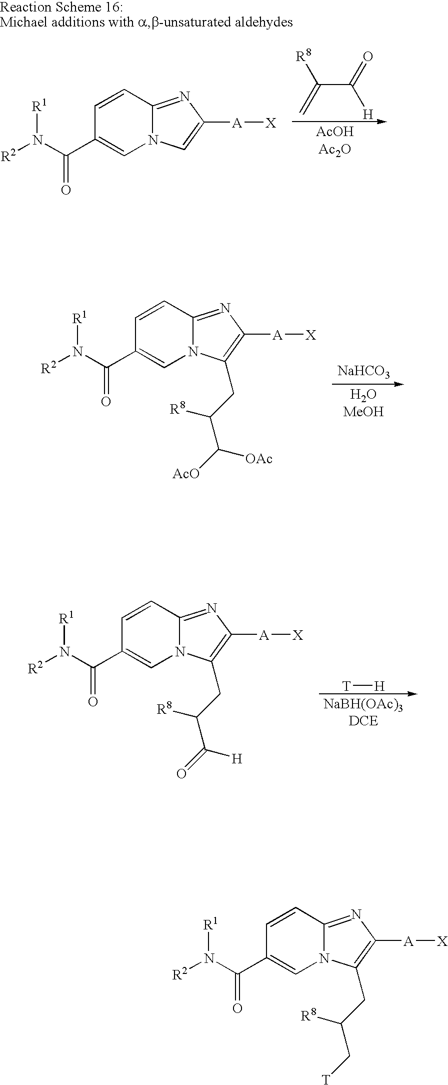

As depicted in Reaction Scheme 16, optionally substituted imidazo[1,2-a]pyridine 6-carboxylic acid amides can be reacted in a Michael addition reaction with α,β-unsaturated aldehydes in a solvent such as a mixture of acetic acid and acetic anhydride at elevated temperature. The reaction may also be carried out in a microwave reactor. The product of this reaction can be treated with a base such as sodium bicarbonate in a suitable solvent like a mixture of water and methanol to yield the corresponding aldehydes which can be subjected to a reductive amination with an amine T-H in the presence of a reducing agent such as sodium triacetoxyborohydride in an appropriate solvent like DCE.

Alternatively, optionally substituted imidazo[1,2-a]pyridine 6-carboxylic acid esters can be used as starting materials. In this case the ester function can be converted to the amide after introduction of the side chain —CH₂CHR⁸CH₂T using the methods described in Reaction Scheme 13.

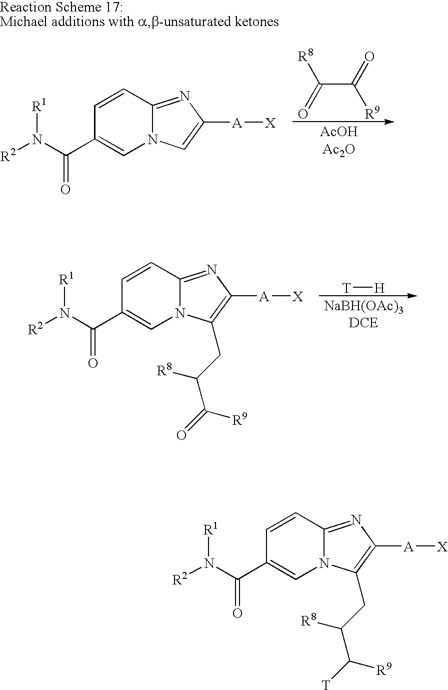

As shown in Reaction Scheme 17, Michael addition of optionally substituted imidazo[1,2-a]pyridine 6-carboxylic acid amides can also be performed with α,β-unsaturated ketones using the reaction conditions described in Reaction Scheme 16. In this case the product of the Michael addition reaction can be directly subjected to the reductive amination reaction.

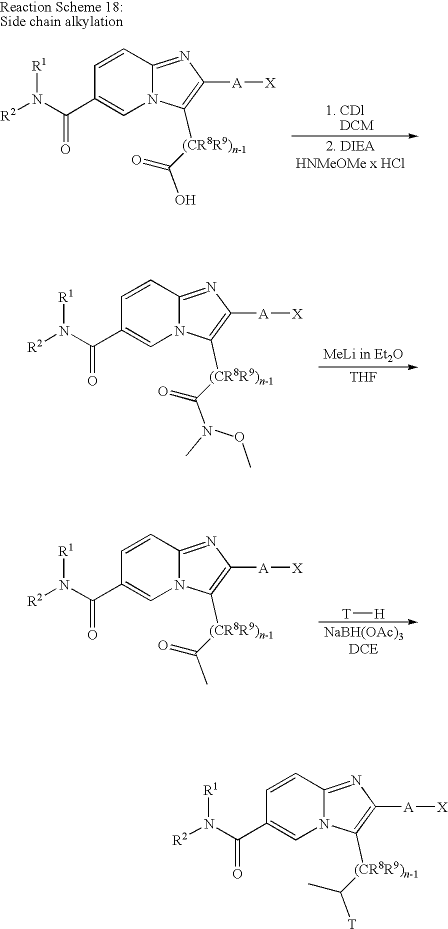

The products from Reaction Scheme 4, optionally substituted imidazo[1,2-a]pyridines bearing a carboxylate function in the side chain can be activated with a reagent such as CDI in an appropriate solvent like DCM and subsequently being reacted with N,O-dimethyl hydroxylamine hydrochloride in the presence of a suitable base such as DIEA. Reaction of the product with a reagent such as methyllithium in a suitable solvent such as THF or diethyl ether leads to the corresponding ketones which can be reductively aminated with an amine T-H in the presence of a reducing agent such as sodium triacetoxyborohydride in an appropriate solvent like DCE.

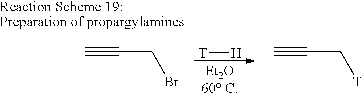

Propargylamines can be prepared as depicted in Reaction Scheme 19. Propargylbromide is reacted with an optionally substituted amine T-H in a solvent like diethyl ether at elevated temperature to yield the desired product.

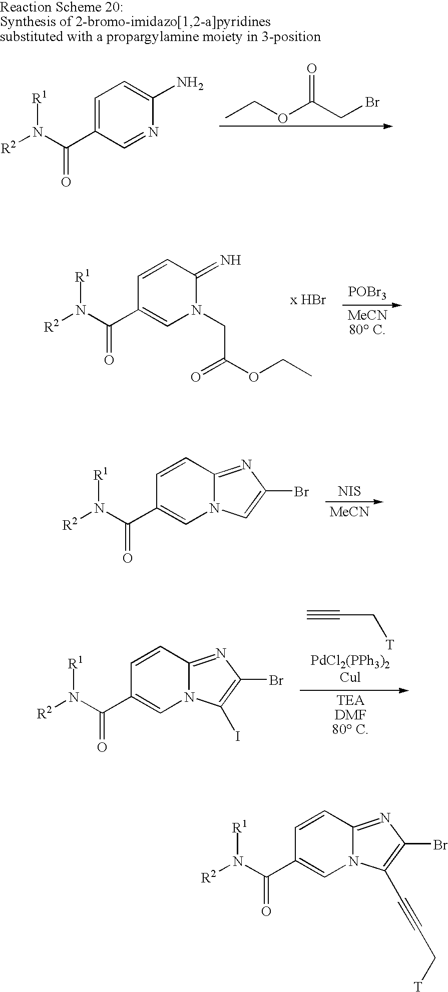

Reaction Scheme 20 shows how optionally substituted 2-amino-pyridine-5-carboxylic acid amides can be reacted with neat ethyl bromoacetate. The products of this reaction can be cyclized using a reagent such as phosphoryl bromide in a suitable solvent like acetonitrile at an appropriate temperature. Alternatively, the cyclization can also be accomplished in neat phosphoryl chloride at 120° C. to yield 2-chlorosubstituted heterocycles. 2-Bromo-imidazo[1,2-a]pyridines, can be iodinated in 3-position using a reagent such as NIS in a suitable solvent like acetonitrile. Reaction with propargylamines, in the presence of a catalyst such as bis(triphenylphosphine)-palladium(II) dichloride, a copper salt like copper(I) iodide and a suitable base like TEA in an appropriate solvent such as DMF at a given temperature leads to the 3-alkinylated products.

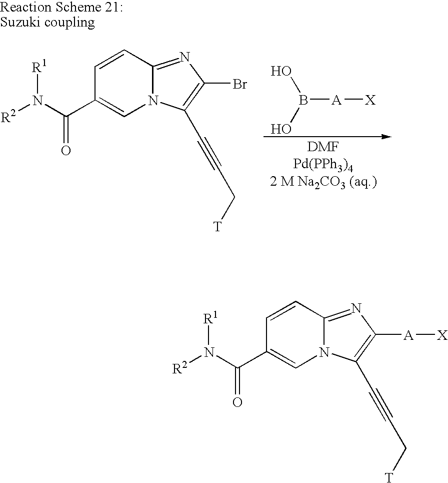

As shown in Reaction Scheme 21, optionally substituted 2-bromo-imidazo[1,2-a]pyridines can be subjected to a Suzuki coupling reaction with boronic acids (HO)₂B-A-X or analogues boronic esters using a catalyst such as tetrakis(triphenylphosphine)palladium(0) in the presence of a base such as aqueous sodium carbonate solution in a suitable solvent like DMF at an appropriate temperature to provide the target compounds.

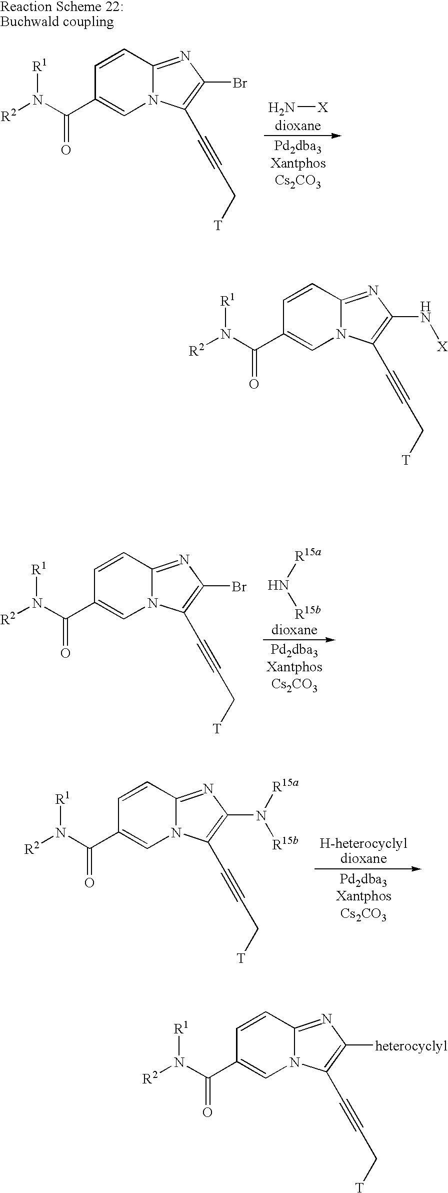

Optionally substituted 2-bromo-imidazo[1,2-a]pyridines can also be reacted with amines under Buchwald conditions, as shown in Reaction Scheme 22. The starting material can be reacted with optionally substituted amines, H₂N—X, HNR^{15a}R^{15b} or H-heterocyclyl (e.g. pyrrolidine, piperidine, morpholine and the like) in the presence of a palladium source like tris(dibenzylideneacetone)dipalladium(0) and a ligand such as 4,5-bis(diphenylphosphino)-9,9-dimethylxanthene in an appropriate solvent like dioxane in the presence of a suitable base such as cesium carbonate at a given temperature.

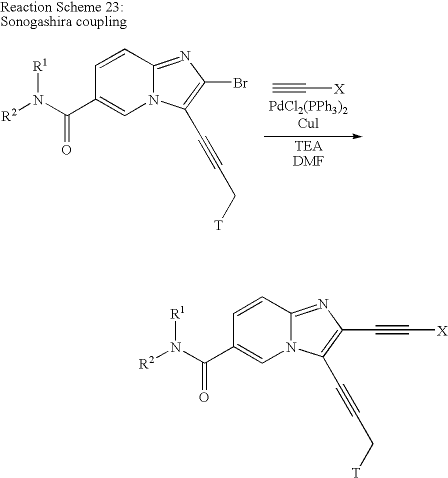

As shown in Reaction Scheme 23, optionally substituted 2-bromo-imidazo[1,2-a]pyridines can be reacted in a Sonogashira coupling with optionally substituted alkynes in the presence of a catalyst such as bis(triphenylphosphine)palladium(II) dichloride, a reagent such as copper(I) iodide and a base like TEA in a suitable solvent like DMF at an appropriate temperature to yield the target compounds.

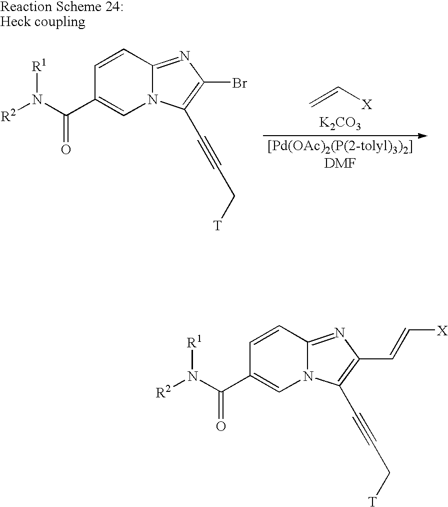

The intermediates from Reaction Scheme 20, optionally substituted 2-bromo-imidazo[1,2-a]pyridines can alternatively be subjected to a Heck coupling. Reaction with an optionally substituted alkene in the presence of a catalyst like [Pd(OAc)₂(P(2-tolyl)₃)₂] (Herrmann, W. A. et al., Angew. Chem. 1995, 107, 1989-1992) and a base such as potassium carbonate in a suitable solvent like DMF at an appropriate temperature yields the target compounds.

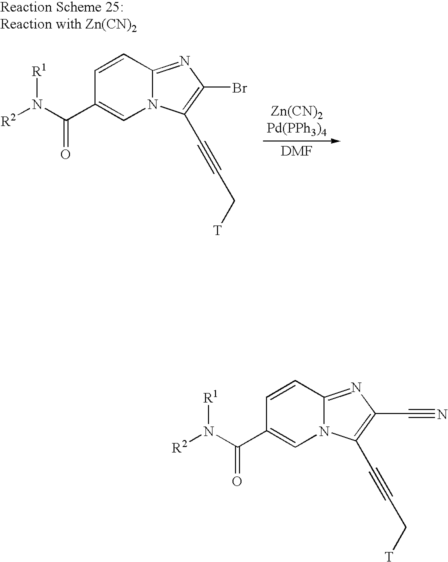

As shown in Reaction Scheme 25, optionally substituted 2-bromo-imidazo[1,2-a]pyridines can also be reacted with a reagent like zinc cyanide in the presence of a catalyst such as tetrakis-(triphenylphosphine)palladium(0) in a suitable solvent like DMF at an appropriate temperature to yield the corresponding nitriles.

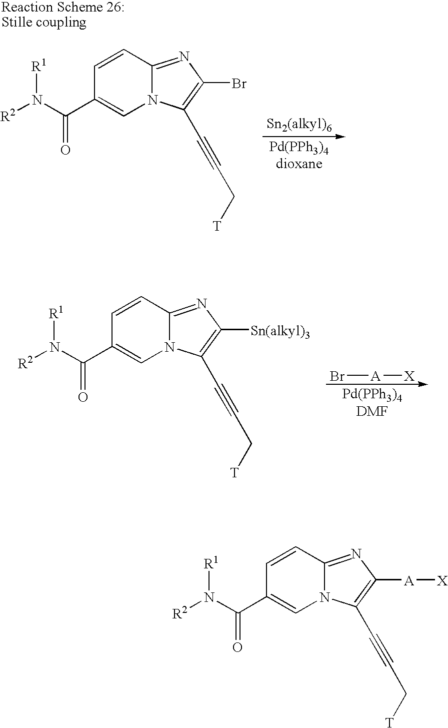

As shown in Reaction Scheme 26, optionally substituted 2-bromo-imidazo[1,2-a]pyridines can be reacted with a reagent like hexaalkylditin in the presence of a catalyst such as tetrakis-(triphenylphosphine)palladium(0) in a suitable solvent like 1,4-dioxane at an appropriate temperature to yield the corresponding 2-trialkylstannyl-imidazo[1,2-a]pyridines. Subsequent reaction of these products with optionally substituted arylhalides or heteroarylhalides in the presence of a catalyst such as tetrakis-(triphenylphosphine)palladium(0) in a solvent like DMF at a given temperature leads to the target compounds.

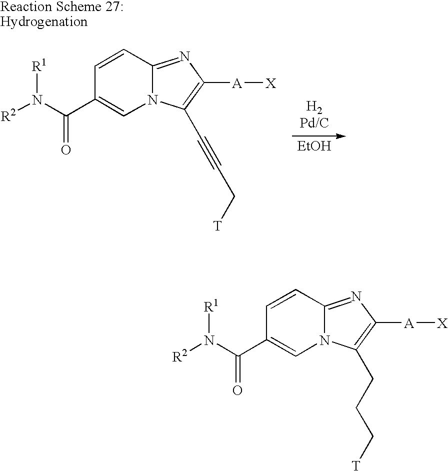

As shown in Reaction Scheme 27, optionally substituted 3-alkinyl-imidazo[1,2-a]pyridines can be hydrogenated in the presence of a catalyst such as palladium on charcoal in an appropriate solvent like ethanol to yield the target compounds.

### Analytical LC-MS

The compounds of the present invention according to formula (I) were analyzed by analytical LC-MS. The conditions are summarized below.

### Analytical Conditions Summary:

LC10Advp-Pump (Shimadzu) with SPD-M10Avp (Shimadzu) UV/Vis diode array detector and QP2010 MS-detector (Shimadzu) in ESI+ modus with UV-detection at 214, 254 and 275 nm,
Column: Waters XTerra MS C18, 3.5 μm, 2.1*100 mm, linear gradient with acetonitrile in water (0.15% HCOOH)
Flow rate of 0.4 ml/min;
Mobile Phase A: water (0.15% HCOOH)
Mobile Phase B: acetonitrile (0.15% HCOOH)

Methods are:

### A:

start concentration 10% acetonitrile (0.15% HCOOH)

| | | |
|---|---|---|
| 10.00 | B. Conc | 60 |
| 11.00 | B. Curve | 2 |
| 12.00 | B. Conc | 99 |
| 15.00 | B. Conc | 99 |
| 15.20 | B. Conc | 10 |
| 18.00 | Pump STOP | |

### Gradient B:

start concentration 1% acetonitrile (0.15% HCOOH)

| | | |
|---|---|---|
| 9.00 | B. Conc | 30 |
| 10.00 | B. Curve | 3 |
| 12.00 | B. Conc | 99 |
| 15.00 | B. Conc | 99 |
| 15.20 | B. Conc | 1 |
| 18.00 | Pump STOP | |

The following describes the detailed examples of the invention which can be prepared via the reaction schemes 1 to 27.

TABLE 1

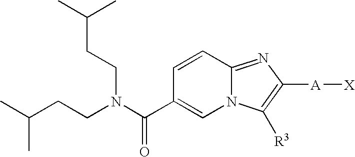

| | | | | | | MS | |
|---|---|---|---|---|---|---|---|
| | | | | HPLC | | MW | |
| | | | | t_{R} | | (calc.) | [M + H]⁺ |
| No. | salt | A—X | R³ | (min) | method | free base | (found) |
| 1 | 2 × HCOOH | 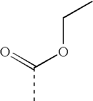 | 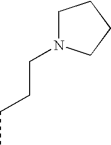 | 6.20 | A | 484.68 | 485 |
| 2 | 2 × HCOOH | 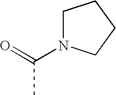 | 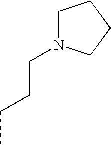 | 6.97 | A | 509.73 | 510 |
| 3 | 2 × HCOOH | 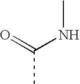 | 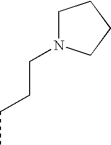 | 6.18 | A | 469.67 | 470 |
| 4 | 2 × HCOOH | 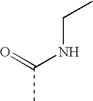 | 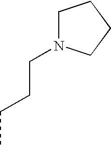 | 6.87 | A | 483.70 | 484 |
| 5 | HCOOH | 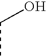 | 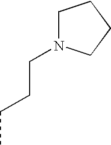 | 4.41 | A | 442.64 | 443 |
| 6 | 2 × HCOOH | 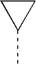 | 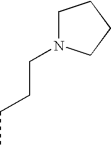 | 3.70 | A | 452.68 | 453 |
| 7 | HCOOH | 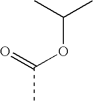 | 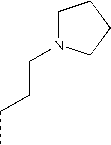 | 6.64 | A | 498.71 | 500 |
| 8 | 2 × HCOOH | 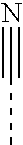 | 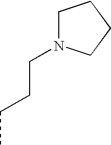 | 6.39 | A | 437.63 | 439 |
| 9 | HCOOH | 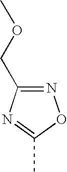 | 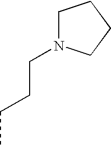 | 6.80 | A | 524.71 | 525 |
| 10 | HCOOH | 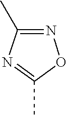 | 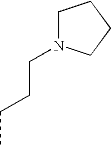 | 6.62 | A | 494.68 | 495 |
| 11 | HCOOH | 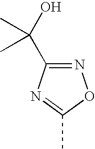 | 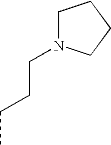 | 6.46 | A | 538.73 | 539 |
| 12 | 2 × HCOOH | 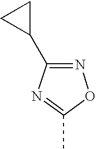 | 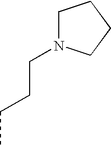 | 6.92 | A | 520.72 | 521 |
| 13 | HCOOH | 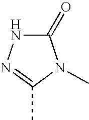 | 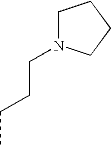 | 6.02 | A | 509.69 | 511 |
| 14 | — | 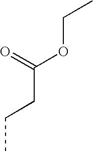 | 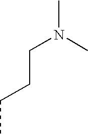 | 4.82 | A | 486.70 | 487 |
| 15 | 2 × HCOOH | 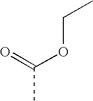 | 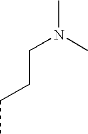 | 5.60 | A | 458.64 | 459 |
| 16 | 2 × HCOOH | 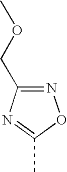 | 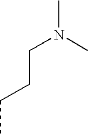 | 6.40 | A | 498.67 | 500 |
| 17 | HCOOH | 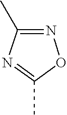 | 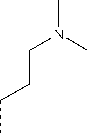 | 6.38 | A | 468.64 | 470 |
| 18 | HCOOH | 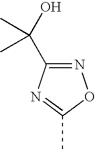 | 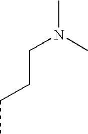 | 6.19 | A | 512.69 | 513 |
| 19 | 2 × HCOOH | 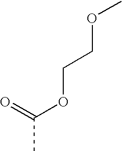 | 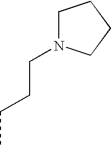 | 6.07 | A | 514.71 | 516 |
| 20 | HCOOH | 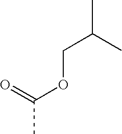 | 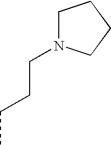 | 7.03 | A | 512.73 | 514 |
| 21 | — | 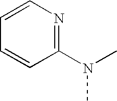 | 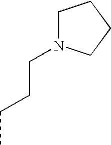 | 4.49 | A | 518.75 | 519 |
| 22 | — | 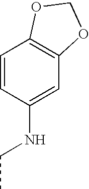 | 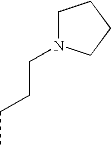 | 5.32 | A | 561.77 | 562 |
| 23 | — | 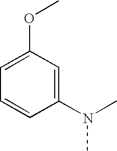 | 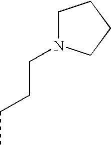 | 6.58 | A | 547.78 | 548 |
| 24 | — | 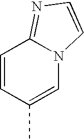 | 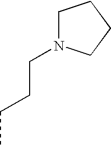 | 4.78 | A | 528.74 | 529 |
| 25 | HCOOH | 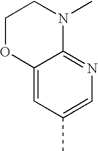 | 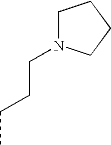 | 4.73 | A | 560.78 | 561 |
| 26 | — | 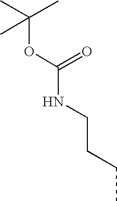 | 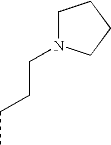 | 5.48 | A | 569.83 | 570 |
| 27 | 2 × HCOOH | 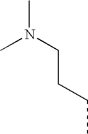 | 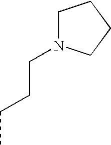 | 6.71 | B | 497.77 | 498 |
| 28 | 3 × HCl | 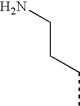 | 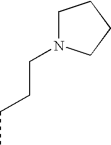 | 2.99 | A | 469.71 | 470 |
| 29 | 2 × HCOOH | 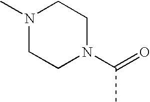 | 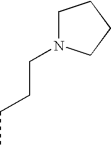 | 8.44 | B | 538.78 | 539 |

The following examples are provided to illustrate the invention and are not limiting the scope of the invention in any manner.

### Synthesis of Examples 1

### Intermediate 1a)

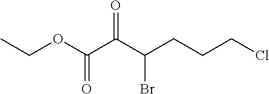

Under argon atmosphere, to a stirred suspension of copper(II) bromide (6.03 g) in ethyl acetate (125 ml) was added ethyl-6-chloro-2-oxohexanoate (5.20 g) in chloroform (125 ml). The resulting mixture was stirred at reflux temperature overnight. Copper(II) bromide (6.03 g) was added and stirring at reflux temperature was continued overnight. The copper salt was filtered on Celite, and the liquid layer was evaporated to dryness.

### Intermediate 1b)

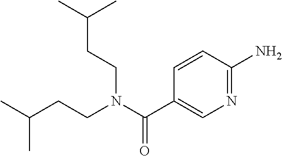

6-Aminonicotinic acid (20.0 g) was dissolved in DMF (300 ml) and DCM (75 ml) and treated with diisopentylamine (36.0 ml), EDC (34.0 g), HOBt (26.0 g) and N,N-diisopropylethylamine (30.0 ml) in this order, stirred at 50° C. overnight, and completely evaporated. The residue was re-dissolved in ethyl acetate and washed with brine, saturated sodium bicarbonate solution and brine. The organic layer was dried over sodium sulfate, filtered, and evaporated. The crude product was purified by column chromatography.

### Intermediate 1c)

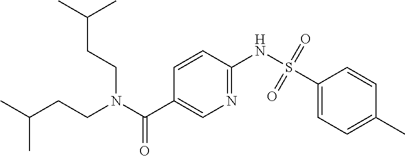

Intermediate 1b) (9.27 g) was dissolved in dry pyridine (150 ml) under Ar and p-toluensulfonylchloride (7.72 g) was added. The reaction mixture was heated at 85° C. overnight. The solvent was removed under reduced pressure, the residue taken up in water and stirred for 5 h. The beige precipitate which formed was filtered off, washed twice with water and dried under high vacuum.

### Intermediate 1d)

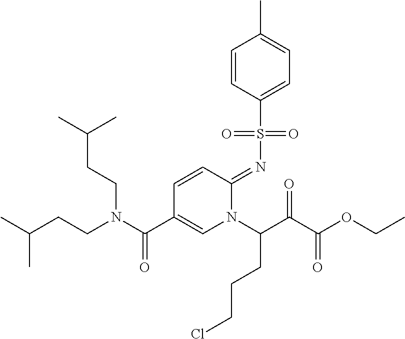

At 50° C. to a stirring suspension of intermediate 1a) (3.94 g) in MeCN (150 ml) was added DIEA (5308 μl). The obtained solution was stirred for 15 min, then intermediate 1c) (6.56 g) in MeCN (150 ml) was added. The obtained solution was stirred at 50° C. for 2 days. Solvents were removed under reduced pressure and the product was purified by flash chromatography.

### Intermediate 1e)

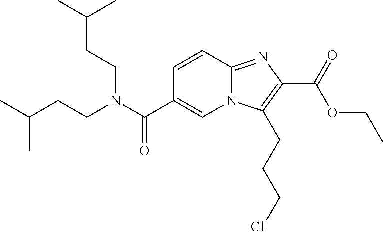

To a stirring solution of intermediate 1d) (4.25 g) in DCE (85 ml) was added trifluoroacetic anhydride (15 ml) and the reaction mixture was stirred at reflux for 3 hours. Volatiles were removed and the residue was taken up in DCM (300 ml) and saturated aqueous Na₂CO₃ solution (150 ml, pH˜11). After phase separation, the organic layer was extracted again with saturated aqueous Na₂CO₃ solution (150 ml). The combined aqueous layer was extracted twice with DCM (100 ml each). The combined organic layer was washed with brine, dried over Na₂SO₄, filtered and volatiles were removed under reduced pressure.

### Example 1

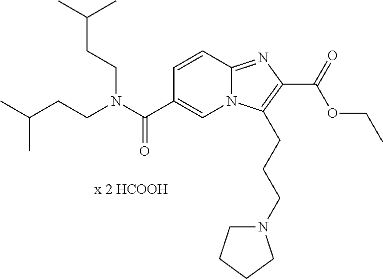

To a stirring solution of intermediate 1e) (2.84 g) in MeCN (75 ml) was added pyrrolidine (5268 μl) and the reaction mixture was stirred at 50° C. for 2 d. Volatiles were removed under reduced pressure and the residue purified by column chromatography. The material for biological testing was subsequently purified with preparative HPLC-MS.

### Example 2

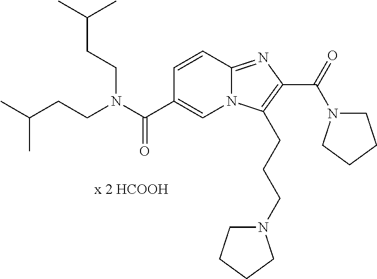

Intermediate 1e) (118 mg) was dissolved in acetonitrile (10 ml). Pyrrolidine (188 μl) was added and the reaction mixture was stirred at 75° C. for 4 h. The solvent was removed under reduced pressure. The product was purified with preparative HPLC-MS.

### Synthesis of Example 4

### Intermediate 4a)

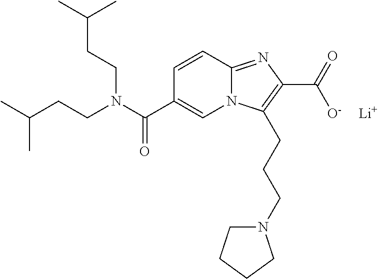

Example 1 (531 mg, free base) was dissolved in THF (12.5 ml) and cooled to 0° C. A solution of lithium hydroxide monohydrate (126 mg) in water (2.5 ml) was added at this temperature. The reaction mixture was stirred for 30 minutes at 0° C. and at RT over night. A second aliquot of lithium hydroxide monohydrate (63 mg) in water (2.5 ml) was added and the reaction mixture was stirred at RT over night. Volatiles were removed and the obtained salt dried in a dessicator under vacuum for three days.

### Intermediate 4b)

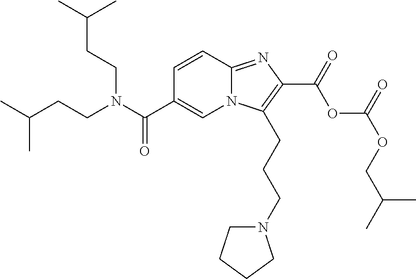

Intermediate 4a) (655 mg) and N-methylmorpholine (550 μl) were dissolved in THF (49 ml) and cooled to an internal temperature of −40° C. Then isobutylchloroformate (649 μl) was added and the reaction mixture was stirred at −40° C. for 2 h. The reaction mixture (colloidal suspension) was used as such in next step.

### Example 4

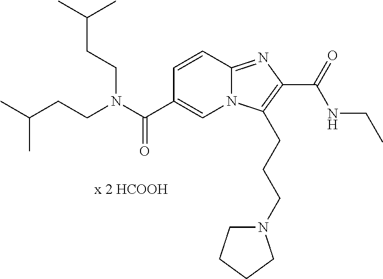

70% Ethylamine in water (159 μl) was added dropwise to a solution of intermediate 4b) (10 ml) and then the reaction mixture was left stirring at −40° C. to −45° C. for 2 hours and then at −20° C. for 1 h. A second portion of 70% ethylamine in water (80 μl) was added and the reaction mixture was stirred at 0° C. overnight. Volatiles were removed and the residue was taken up with methanol and filtered. Solvent was removed and the crude product purified using preparative HPLC.

### Synthesis of Example 5

### Example 5

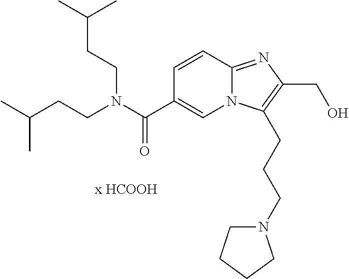

A solution of sodium borohydride (45.4 mg) in water (1.5 ml) was added dropwise to a solution of intermediate 4b) (10 ml) and the reaction mixture was left stirring at −40° C. to −45° C. for 2 hours. The reaction mixture was then stirred at 0° C. for 1 h. Sodium borohydride (45.4 mg) was added and the reaction mixture was stirred at 0° C. overnight. Volatiles were removed and the residue was taken up with methanol and filtered. Solvent was removed and the crude product purified using preparative HPLC.

### Synthesis of Examples 6

### Intermediate 6a)

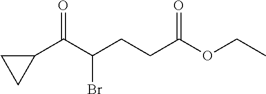

Under argon atmosphere, to a stirred suspension of copper(II) bromide (2.42 g) in ethyl acetate (20 ml) was added ethyl 5-cyclopropyl-5-oxovalerate (1.00 g) in chloroform (20 ml). The resulting mixture was stirred at reflux temperature overnight. The reaction mixture was filtered through Celite and volatiles were removed. The residue was taken up with DCM and washed twice with water. The aqueous layer was extracted back with DCM. The combined organic layer was washed with brine, dried over sodium sulfate, filtered and the solvent was carefully removed.

### Intermediate 6b)

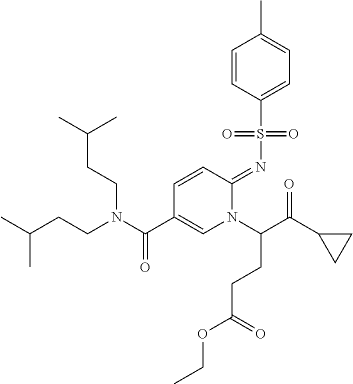

At 50° C., to a stirring suspension of intermediate 6a) (1.10 g) of in MeCN (35 ml) was added DIEA (1529 μl). The obtained solution was stirred for 15 min, then intermediate 1c) (1.89 g) in MeCN (35 ml) was added. The obtained solution was stirred at 50° C. for 2 days. Solvents were removed under reduced pressure and the product was purified by flash chromatography.

### Intermediate 6c)

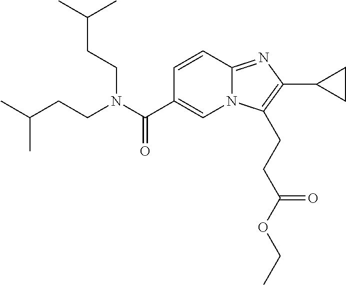

To a stirring solution of intermediate 6b) (860 mg) in DCE (45 ml) was added trifluoroacetic anhydride (5 ml) and the reaction mixture was stirred at reflux for 3 hours. Volatiles were removed and the residue was taken up in DCM (50 ml) and saturated aqueous Na₂CO₃ solution (100 ml, pH˜11). After phase separation, the aqueous layer was extracted twice with DCM (5 ml each). The combined organic layer was washed with brine, dried over Na₂SO₄, filtered and volatiles were removed under reduced pressure. The crude product was purified by flash chromatography.

### Intermediate 6d)

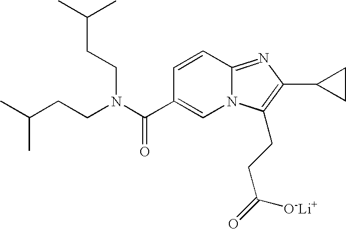

Intermediate 6c) was dissolved in THF (25 ml) and cooled to 0° C. A solution of LiOH monohydrate (126 mg) in water (5 ml) was added at this temperature, the ice-bath was removed after 30 minutes and the reaction mixture was stirred at RT overnight. Volatiles were distilled off and water was co-evaporated with toluene. The product was dried in a dessicator under vacuum for 24 h.

### Intermediate 6e)

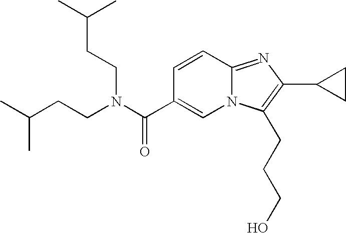

Intermediate 6d) and N-methylmorpholine (121 μl) were dissolved in THF (20 ml) and cooled to an internal temperature of −20° C. Isobutylchloroformate (195 μl) was added at this temperature and the reaction mixture was stirred at −20° C. for 90 minutes. A solution of sodium borohydride (91 mg) in water (2 ml) was added dropwise, the reaction mixture was allowed to warm to RT, and stirred overnight. The reaction mixture was concentrated in vacuum. The residue was diluted with DCM, transferred into an extraction funnel and washed with saturated aqueous Na₂CO₃ solution and brine. All aqueous layers were extracted with DCM. The combined organic layer was washed with brine, dried over Na₂SO₄ and volatiles were removed.

### Intermediate 6f)

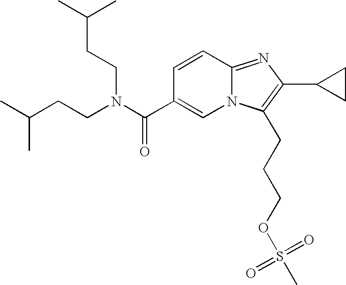

Intermediate 6e) (451 mg) was dissolved in dry THF (15 ml) and dry DCM (15 ml) and triethylamine (211 μl) was added followed by methanesulfonyl chloride (116 μl). The reaction mixture was stirred at RT for 3 h. Volatiles were removed and the residue was taken up with DCM (50 ml) and extracted twice with 1M aqueous NaHCO₃ solution (25 ml each). The aqueous layer was extracted back with DCM (25 ml) and the combined organic layer was washed with brine, dried over Na₂SO₄, filtered and the solvent was removed under reduced pressure.

### Example 6

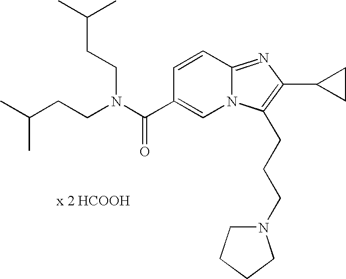

Pyrrolidine (417 μl) was added to intermediate 6f) (300 mg) in MeCN (10 ml) and the reaction mixture was stirred at 50° C. overnight. Volatiles were removed and the residue was take up with DCM, washed with saturated aqueous Na₂CO₃ solution and brine, dried over Na₂SO₄, filtered and volatiles were removed. The crude product was purified with preparative HPLC-MS.

### Synthesis of Example 8

### Intermediate 8a)

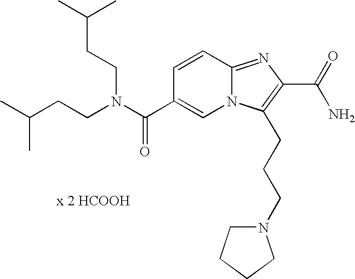

Intermediate 4a) (29 mg) was dissolved in a mixture of DMF (15 ml) and DCM (5 ml). 25% Aqueous ammonia (924 μl), EDC (230 mg), HOBt (184 mg) and DIEA (209 μl) were added. The reaction was stirred in a sealed tube at 50° C. for 3 d. A second aliquot of 25% aqueous ammonia (924 μl) was added and stirring at 50° C. was continued overnight. Volatiles were removed and the residue was taken up with DCM, and washed twice with saturated aqueous Na₂CO₃ solution. The combined aqueous layer was extracted back with DCM. The combined organic layer was washed with brine. Volatiles were removed and the crude product was purified by preparative HPLC-MS.

### Example 8

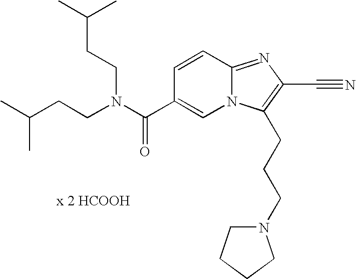

To a stirring solution of intermediate 8a) (29 mg) in THF (2 ml) was added pyridine (100 μl). The reaction mixture was stirred at RT for 30 min and trifluoroacetic anhydride (8.44 μl) was added at 0° C. The reaction mixture was stirred at RT overnight. Additional trifluoroacetic anhydride (8.44 μl) was added and the reaction mixture stirred at RT overnight. A third aliquot of trifluoroacetic anhydride (8.44 μl) was added and the reaction mixture stirred at RT for 3 d. Volatiles were removed and the residue taken up in ethyl acetate. The organic layer was extracted twice with saturated aqueous Na₂CO₃ solution. The combined aqueous layer was extracted with ethyl acetate. The combined organic layer was washed with brine, dried over Na₂SO₄, filtered and volatiles were removed. The crude product was purified with preparative HPLC-MS.

### Synthesis of Example 13

### Intermediate 13a)

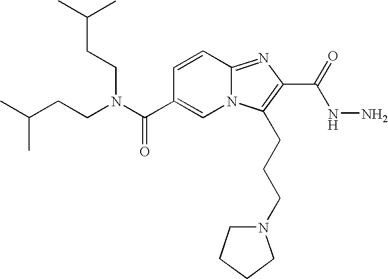

A mixture of example 1 (485 mg, free base) and hydrazine monohydrate (121.5 μl) in ethanol (10 ml) was kept under reflux for 2 days. A second aliquot of hydrazine monohydrate (121.5 μl) was added and the reaction mixture was heated under reflux for additional three days. Volatiles were removed under reduced pressure.

### Intermediate 13b)

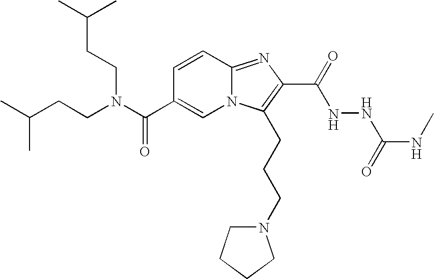

At RT, under argon atmosphere, to a stirred solution of intermediate 13a) (240 mg) in THF (15 ml) was added methyl isocyanate (44.2 μl) dropwise. The reaction mixture was stirred at RT overnight. Addition of diethyl ether (15 ml) led to precipitation of the product in form of a beige solid which was filtered off. A second batch of product was obtained by concentration of the filtrate. The crude product was used for the next step without purification.

### Example 13

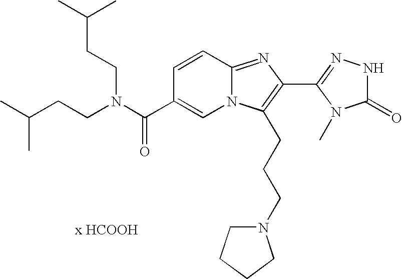

Intermediate 13b) (251 mg) was suspended in 1M aqueous sodium hydroxide solution (595 μl) and the reaction mixture was heated to 120° C. for 60 minutes using microwave irradiation. 1M Aqueous sodium hydroxide solution (100 μd) was added and the reaction heated to 120° C. for another 30 min. A third aliquot of 1M aqueous sodium hydroxide solution (100 μl) was added and the reaction heated to 120° C. for another 30 min period. The reaction mixture was diluted with saturated aqueous sodium bicarbonate solution and extracted with DCM. The organic layer was evaporated under reduced pressure. The crude product was purified with preparative HPLC-MS.

### Synthesis of Example 14

### Intermediate 14a)

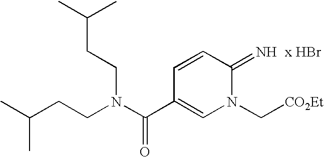

A solution of intermediate 1b) (10.0 g) in ethyl bromoacetate (25 ml) was stirred at 22° C. for 16 h. The reaction mixture was carefully evaporated under high vacuum (0.1 mbar/50° C.), co-evaporated with toluene (5×100 ml), and dried under high vacuum to give the crude product as brown solid, which was used in the next step without any further purification.

### Intermediate 14b)

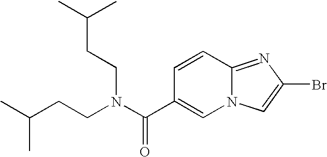

At 22° C., a solution of phosphorus(V) oxybromide (20.7 g) in acetonitrile (40 ml) was added dropwise over a time period of 20 min to a solution of the crude intermediate 14a) (17.1 g) in acetonitrile (110 ml). The reaction mixture was transferred to a pre-heated oil bath (80° C.), and stirred 18 h at this temperature. The reaction mixture was cooled to 22° C., diluted with ethyl acetate (350 ml) and slowly poured onto a mixture of ice and saturated sodium bicarbonate solution (300 ml). After separation, the organic layer was washed with saturated sodium bicarbonate solution (3×200 ml). The combined aqueous layer was extracted with ethyl acetate (4×150 ml). The combined organic extract was washed with brine (400 ml), dried over Na₂SO₄, filtered, and evaporated. The crude material was purified by flash chromatography.

### Intermediate 14c)

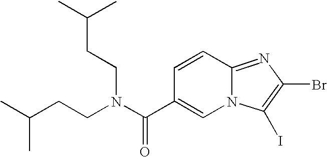

A solution of intermediate 14b) (4.47 g) in acetonitrile (90 ml) was treated with N-iodosuccinimide (2.90 g) and stirred in the absence of light at 22° C. for 1 h. The reaction mixture was diluted with diethyl ether (150 ml) and washed with 1M aqueous sodium thiosulfate solution (3×100 ml). The combined aqueous layer was washed with diethyl ether (2×80 ml). The combined organic extract was washed with water (100 ml) and brine (100 ml), dried over sodium sulfate, filtered, and evaporated to give the crude product, which was used in the next step without any further purification.

### Intermediate 14d)

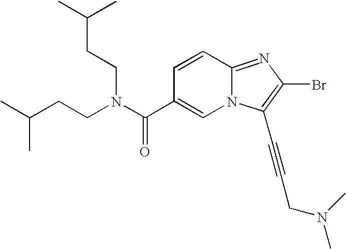

A degassed mixture of intermediated 14c) (300 mg), Pd(PPh₃)₂Cl₂ (21 mg), copper(I) iodide (17 mg), and triethylamine (0.41 ml) in DMF (10 ml) was treated with 1-dimethylamino-2-propyne (0.25 ml) and stirred for 2.5 h at 80° C. The reaction mixture was diluted with diethyl ether (35 ml) and washed with water (3×25 ml). The combined aqueous layer was extracted with diethyl ether (2×20 ml). The combined organic extract was washed with brine (50 ml), dried over sodium sulfate, filtered, and evaporated. The crude product was purified by flash chromatography.

### Intermediate 14e)

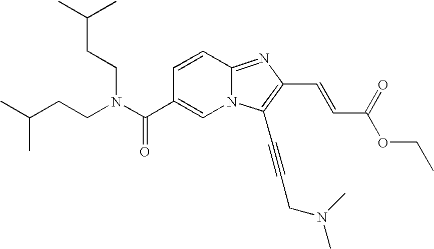

A degassed mixture of intermediate 14d) (50 mg), [Pd(OAc)₂(P(2-tolyl)₃)₂] (Herrmann, W. A. et al., Angew. Chem. 1995, 107, 1989-1992) (10 mg) and anhydrous K₂CO₃ (60 mg) in DMF (2 ml) was treated with ethyl acrylate (0.30 ml) and stirred for 19 h at 130° C. The reaction mixture was diluted with diethyl ether (35 ml) and washed with water (3×25 ml). The combined aqueous layer was extracted with diethyl ether (2×20 ml). The combined organic extract was washed with brine (50 ml), dried over sodium sulfate, filtered, and evaporated. The crude product was purified by flash chromatography.

### Example 14

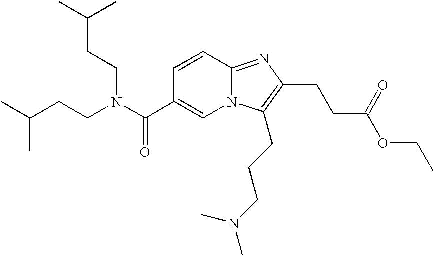

A degassed mixture of intermediate 14e) (17 mg) and 10% Pd/C (6 mg) in EtOH (1 ml) was hydrogenated at atmospheric pressure for 90 min at 22° C. The reaction mixture was filtered through a pad of Celite and washed with MeOH (30 ml). The combined filtrate and washings were evaporated and the crude product was purified by flash chromatography.

### Synthesis of Example 15

### Example 15

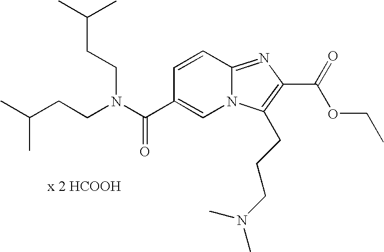

To a stirring solution of intermediate 1e) (3.10 g) in MeCN (80 ml) was added a 2N solution of dimethylamine in THF (34.4 ml) and the reaction mixture was stirred at 50° C. over night. Volatiles were removed under reduced pressure and the residue purified by column chromatography.

The material for biological testing was subsequently purified with preparative HPLC-MS.

### Synthesis of Example 17

### Intermediate 17a)

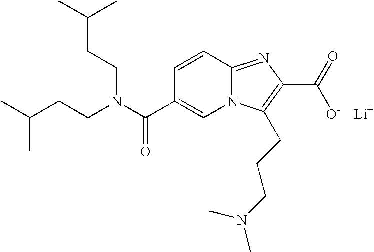

Example 15 (3.00 g) was dissolved in THF (60 ml) and cooled to 0° C. A 2N solution of lithium hydroxyde in water (9.8 ml) was added at this temperature. The reaction mixture was stirred for 30 minutes at 0° C. and at RT over night. Volatiles were removed and the obtained powder dried under vacuum overnight.

### Intermediate 17b)

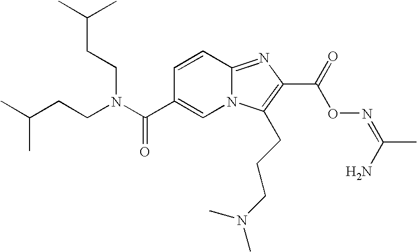

Intermediate 17a) (400 mg) was dissolved in THF (45 ml). N-Methylmorpholine (503 μl) was added and the mixture cooled to −40° C. before isobutylchloroformate (594 μl) was added and the mixture left to stir at −40° C. for 2 h. N-Hydroxyacetamidine (270 mg) was added and the reaction mixture stirred for another 2 h at −40° C. Solvents were removed under reduced pressure and the crude material taken to the next step without purification.

### Example 17

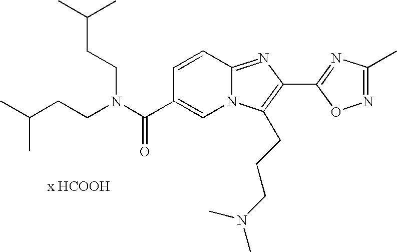

Intermediate 17b) (581 mg) was dissolved in pyridine (16 ml) and the mixture heated to 95° C. overnight. Solvents were removed under reduced pressure and the crude product purified by preparative HPLC-MS.

### Synthesis of Example 19

### Example 19

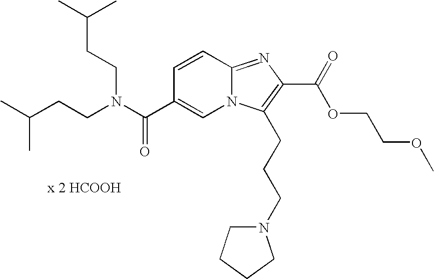

At RT, to a stirring solution of intermediate 4a) (259 mg) in 2-methoxyethanol (5 ml) was added thionyl chloride (182 μl) followed by DMF (20 μl). The reaction mixture was stirred at RT overnight and subsequently heated at 80° C. in a sealed tube overnight. A second aliquot of thionyl chloride (182 μl) was added and the reaction mixture was stirred at 80° C. overnight. Volatiles were removed and the crude product was purified by preparative HPLC-MS.

### Synthesis of Example 21

### Intermediate 21a)

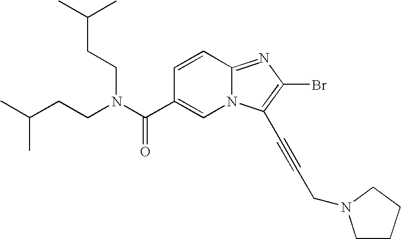

A degassed mixture of intermediate 14c) (6.01 g), Pd(PPh₃)₂Cl₂ (412 mg), copper(I) iodide (336 mg), and triethylamine (8.2 ml) in DMF (185 ml) was treated with N-propargyl pyrrolidine (Biel and DiPierro, J. Am. Chem. Soc. 1958, 80, 4609-4614) (2.9 ml) and stirred at 80° C. for 1 h. The reaction mixture was diluted with diethyl ether (150 ml) and washed with water (3×200 ml). The combined aqueous layer was extracted with diethyl ether (4×150 ml). The combined organic extract was washed with brine (250 ml), dried over sodium sulfate, filtered, and evaporated. The crude product was purified by flash chromatography.

### Intermediate 21b)

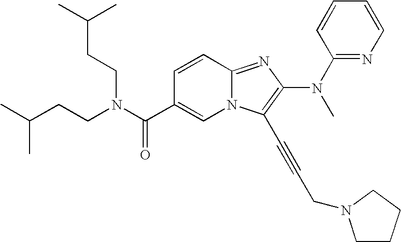

At 22° C., a mixture of intermediate 21a) (50 mg) and 2-(methylamino)pyridine (21 μl) in 1,4-dioxane (1 ml) was subjected to 3 cycles of evacuation/backfilling with Ar procedure and then treated successively with Pd₂ dba₃ (9.4 mg) and 4,5-bis(diphenylphosphino)-9,9-dimethylxanthene (17.8 mg). The evacuation/backfilling with Ar procedure was repeated three times and cesium carbonate (67 mg, freshly dried with a heat gun under high vacuum before use) was added in one portion. The evacuation/backfilling with Ar procedure was repeated three times. The reaction mixture was transferred to a pre-heated oil bath (110° C.) and stirred in a sealed tube at this temperature for 4.5 h. The mixture was diluted with ethyl acetate (20 ml) and washed with water (1×25 ml). The aqueous layer was extracted with ethyl acetate (1×15 ml). The combined organic extracts were washed with brine (30 ml), dried over sodium sulfate, filtered, and evaporated. The crude product was purified by column chromatography.

### Example 21

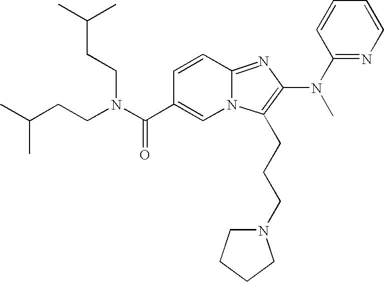

A degassed mixture of intermediate 21b) (35 mg) and 10% Pd/C (14.5 mg) in EtOH (3 ml) was hydrogenated at atmospheric pressure for 5.5 h at 22° C. The reaction mixture was filtered through a pad of Celite and washed with MeOH (20 ml). The combined filtrate and washings were evaporated and the crude product was purified by flash chromatography.

### Synthesis of Example 22

### Intermediate 22a)

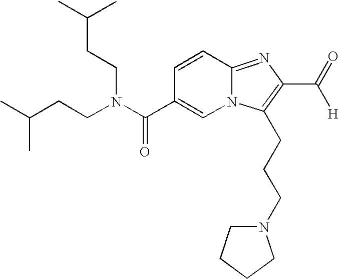

At −78° C. and under Ar atmosphere, a solution of example 1 (free base) (350 mg) in THF (5 ml) was treated dropwise with 1M solution of DIBAL-H in hexanes (1.3 ml). The reaction mixture was stirred for 4 h at −78° C., diluted with DCM (20 ml), treated with 1M aqueous HCl (2 ml) solution and washed with saturated aqueous NaHCO₃ solution (15 ml). The aqueous layer was washed with DCM (10 ml). The combined organic extract was dried over sodium sulfate, filtered, and evaporated. The crude product was used in the next step without any further purification.

### Example 22

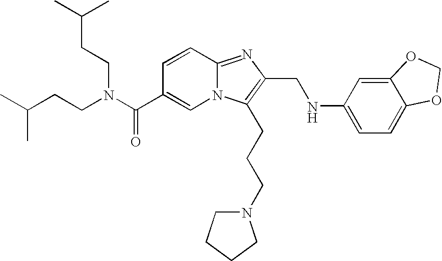

A mixture of crude intermediate 22a) (65 mg) and 3,4-(methylenedioxy)aniline (23 mg) in 1,2-dichloroethane (1.5 ml) was treated with NaBH(OAc)₃ and stirred for 24 h at 22° C. The reaction mixture was diluted with ethyl acetate (25 ml) and washed with saturated aqueous NaHCO₃ solution (2×20 ml). The combined aqueous layer was washed with ethyl acetate (2×15 ml). The combined organic extract was washed with water (30 ml) and brine (30 ml), dried over sodium sulfate, filtered, and evaporated. The desired product was isolated by preparative HPLC-MS.

### Synthesis of Example 25

### Intermediate 25a)

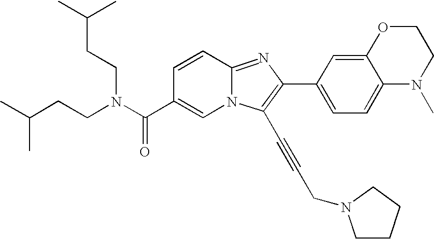

A degassed mixture of intermediate 21a) (200 mg), 4-methyl-7-(4,4,5,5-tetramethyl-1,3,2-dioxaborolan-2-yl)-3,4-dihydro-2H-pyrido[3,2-b][1,4]oxazine (170 mg), and 2M aqueous sodium carbonate solution (0.8 ml) in DMF (1 ml) was treated with Pd(PPh₃)₄ (47 mg). The reaction mixture was transferred to a pre-heated oil bath (110° C.) and stirred at this temperature for 3 h. The mixture was diluted with diethyl ether (50 ml) and washed with water (1×20 ml). The aqueous layer was extracted with diethyl ether (3×20 ml). The combined organic extracts were washed with brine (50 ml), dried over sodium sulfate, filtered, and evaporated. The crude product was purified by column chromatography.

### Example 25

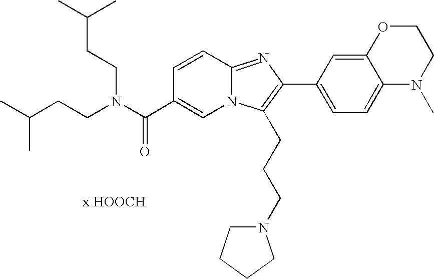

A mixture of intermediate 25a) (100 mg) and 10% Pd/C (38 mg) in ethanol (9 ml) was subjected to 1 atm hydrogen for 5 h. Subsequently, the mixture was filtered through Celite and the solvents were removed under reduced pressure. The crude product was purified by preparative HPLC-MS.

### Biological Assays

### A. Binding Assay

A membrane binding assay is used to identify competitive inhibitors of fluorescence labeled NDP-alpha-MSH binding to HEK293 cell membrane preparations expressing human melanocortin receptors.

The test compound or unlabeled NDP-alpha-MSH is dispensed at varying concentrations to a 384 well microtiter plate. Fluorescence labeled NDP-alpha-MSH is dispensed at a single concentration, followed by addition of membrane preparations. The plate is incubated for 5 h at room temperature.

The degree of fluorescence polarization is determined with a fluorescence polarization microplate reader.

### B. Functional Assay

Agonistic activity of human melanocortin receptors is determined in a homogeneous membrane based assay. Competition between unlabeled cAMP and a fixed quantity of fluorescence labeled cAMP for a limited number of binding sites on a cAMP specific antibody is revealed by fluorescence polarization.

The test compound or unlabeled NDP-alpha-MSH is dispensed at varying concentrations to a 384 well microtiter plate. Membrane preparations from HEK293 cells expressing the human melanocortin receptors are added. After a short preincubation period, an appropriate amount of ATP, GTP and the cAMP antibody is added and the plate is further incubated before the fluorescence labeled cAMP conjugate is dispensed. The plate is incubated for 2 h at 4° C. before it is read on a fluorescence polarization microplate reader. The amount of cAMP produced as a response to a test compound is compared to the production of cAMP resulting from stimulation with NDP-alpha-MSH.

The compounds of the present invention were tested and found to bind to the melanocortin-4 receptor. These compounds were generally found to have IC₅₀ values less than 2 μM. The compounds of the present invention were also tested in the functional assay and found generally not to activate the melanocortin-4 receptor.

TABLE 2
Biological data for the examples of the invention
In the table are listed the IC₅₀ values of the hMC-4R binding
assay and the EC₅₀ values of the functional assay.
The IC₅₀ and EC₅₀ values are grouped in 3 classes: a ≦ 0.1 μM;
b > 0.1 μM and ≦1.0 μM; c > 1.0 μM

| | hMC-4R | | |
|---|---|---|---|
| | binding | hMC-4R | |
| | assay | functional | % activation |
| Example | IC₅₀/nM | assay EC₅₀/nM | functional assay |
| SHU9119 | a | — | 7 |
| NDP-α-MSH | a | a | 100 |
| 1 | a | — | 0 |
| 2 | b | — | 0 |
| 3 | b | — | −12 |
| 4 | b | — | 8 |
| 5 | b | — | −1 |
| 6 | b | — | −8 |
| 7 | b | b | −27 |
| 8 | c | b | −19 |
| 9 | a | b | −41 |
| 10 | a | a | −22 |
| 11 | b | — | −1 |
| 12 | a | — | −7 |
| 13 | b | — | −10 |
| 14 | b | — | 2 |
| 15 | b | — | −1 |
| 16 | b | — | −1 |
| 17 | b | — | 1 |
| 18 | b | b | −39 |
| 19 | a | — | −16 |
| 20 | a | — | −5 |
| 21 | b | — | 6 |
| 22 | a | — | −5 |
| 23 | b | — | −4 |
| 24 | a | — | −10 |
| 25 | a | — | −11 |
| 26 | a | — | −14 |
| 27 | a | b | −51 |
| 28 | a | — | −14 |
| 29 | b | — | −24 |

### C. In Vivo Food Intake Models

### 1. Spontaneous Feeding Paradigm

Food intake in rats is measured after s.c., i.p. or p.o. administration of the test compound (see e.g. Chen, A. S. et al. Transgenic Res 2000 April; 9(2):145-54).

### 2. Models of LPS-Induced Anorexia and Tumor-Induced Cachexia

Prevention or amelioration of anorexia induced by lipopolysaccharide (LPS) administration or cachexia induced by tumor growth is determined upon s.c., i.p. or p.o. administration of test compounds to rats (see e.g. Marks, D. L.; Ling, N. and Cone, R. D. Cancer Res 2001 Feb. 15; 61(4):1432-8).

### D. In Vivo Model for Depression

Forced Swim Test in Mice

### Principle

Animals placed in a container filled with water show periods of increased swimming activity and periods of relative immobility. Clinically active anti-depressants have been found to delay the onset of the first phase of immobility and to reduce the total time of relative immobility. The list of active compounds includes monoamino-oxidase-A (MAO-A) inhibitors such as moclobemide, brofaromine, noradrenaline (NA) uptake inhibitors such as imipramine and amytryptilin, MAO-B inhibitors such as selegiline and tranylcypromine, serotonin uptake inhibitors (SSRI) such as fluoxetine and paroxetine and combined NA/SSRI such as venlafaxine. Benzodiazepines and other types of psychoactive compounds have been found to be inactive in this test (see e.g. Porsolt R. D., Bertin A., Jaffre M. Behavioral despair in rats and mice: strain differences and the effect of imipramine. Eur. J. Pharmacol. 1978, 51: 291-294, Borsini F. and Meli A. Is the forced swimming test a suitable model for revealing antidepressant activity (Psychopharmacol. 1988, 94: 147-160).

### Experimental Procedure

Subjects to be used are male Swiss mice (4-5 weeks old). Animals are randomly assigned to different groups (10 mice per group).

Each animal is placed individually in the water bath where it remains for 6 minutes. The animal is given an accommodation period of 2 minutes. During the subsequent 4 minutes observation period, the duration of the periods of immobility is recorded. In addition, the frequency of the immobility state is also measured. The mouse is considered to be immobile when it passively floats on the water making only small movements to keep its head above the surface.

The water is replaced with clean water after 3 animals tested.

### Drug Administration

Treatment is administered before the test as vehicle or test compound at different doses. Compounds are usually administered by p.o. i.p. or s.c. routes.

### Data Analysis

Analysis of data is performed using ANOVA followed by Fisher's PLSD test as post-hoc test.

### E. In Vitro ADME Assays

### 1. Microsomal Stability

### Experimental Procedure

Pooled human liver microsomes (pooled male and female) and pooled rat liver microsomes (male Sprague Dawley rats) are prepared. Microsomes are stored at −80° C. prior to use.

Microsomes (final concentration 0.5 mg/ml), 0.1 M phosphate buffer pH7.4 and test compound (final substrate concentration=3 μM; final DMSO concentration=0.25%) are pre-incubated at 37° C. prior to the addition of NADPH (final concentration=1 mM) to initiate the reaction. The final incubation volume is 25 μl. A control incubation is included for each compound tested where 0.1 M phosphate buffer pH7.4 is added instead of NADPH (minus NADPH). Two control compounds are included with each species. All incubations are performed singularly for each test compound.

Each compound is incubated for 0, 5, 15, 30 and 45 min. The control (minus NADPH) is incubated for 45 min only. The reactions are stopped by the addition of 50 μl methanol containing internal standard at the appropriate time points. The incubation plates are centrifuged at 2,500 rpm for 20 min at 4° C. to precipitate the protein.

### Quantitative Analysis

Following protein precipitation, the sample supernatants are combined in cassettes of up to 4 compounds and analysed using generic LC-MS/MS conditions.

### Data Analysis

From a plot of the peak area ratio (compound peak area/internal standard peak area) against time, the gradient of the line is determined. Subsequently, half-life and intrinsic clearance are calculated using the equations below:

$$Elimation rate constant ( k ) = ( - gradient )$$

$$Half life ( {t}_{1 / 2} ) ( min ) = \frac{0.693}{k}$$

$$Intrinsic Clearance ( {CL}_{int} ) ( μl \text{/} min \text{/} mg protein ) = \frac{V \times 0.693}{{t}_{1 / 2}}$$

$$where V = Incubation volume μl \text{/} mg microsomal protein .$$

Two control compounds are included in the assay and if the values for these compounds are not within the specified limits the results are rejected and the experiment repeated.

### 2. Hepatocyte Stability

### Experimental Procedure

Suspensions of cryopreserved hepatocytes are used for human hepatocyte stability assay (pooled from 3 individuals). All cryopreserved hepatocytes are purchased from 1n Vitro Technologies, Xenotech or TCS.

Incubations are performed at a test or control compound concentration of 3 μM at a cell density of 0.5×10⁶ viable cells/mL. The final DMSO concentration in the incubation is 0.25%. Control incubations are also performed in the absence of cells to reveal any non-enzymatic degradation.

Duplicate samples (50 μl) are removed from the incubation mixture at 0, 5, 10, 20, 40 and 60 min (control sample at 60 min only) and added to methanol, containing internal standard (100 μl), to stop the reaction.

Tolbutamide, 7-hydroxycoumarin, and testosterone are used as control compounds. The samples are centrifuged (2500 rpm at 4° C. for 20 min) and the supernatants at each time point are pooled for cassette analysis by LC-MS/MS using generic methods.

### Data Analysis

From a plot of ln peak area ratio (compound peak area/internal standard peak area) against time, the gradient of the line is determined. Subsequently, half-life and intrinsic clearance are calculated using the equations below:

$$Elimation rate constant ( k ) = ( - gradient )$$

$$Half life ( {t}_{1 / 2} ) ( min ) = \frac{0.693}{k}$$

$$Intrinsic Clearance ( {CL}_{int} ) ( μl \text{/} min \text{/} million cells ) = \frac{V \times 0.693}{{t}_{1 / 2}}$$

$$where V = Incubation volume ( μl ) / number of cells$$

### 3. Caco-2 Permeability (Bi-Directional)

### Experimental Procedure

Caco-2 cells obtained from the ATCC at passage number 27 are used. Cells (passage number 40-60) are seeded on to Millipore Multiscreen Caco-2 plates at 1×10⁵ cells/cm². They are cultured for 20 days in DMEM and media is changed every two or three days. On day 20 the permeability study is performed.

Hanks Balanced Salt Solution (HBSS) pH7.4 buffer with 25 mM HEPES and 10 mM glucose at 37° C. is used as the medium in permeability studies. Incubations are carried out in an atmosphere of 5% CO₂ with a relative humidity of 95%.

On day 20, the monolayers are prepared by rinsing both basolateral and apical surfaces twice with HBSS at 37° C. Cells are then incubated with HBSS in both apical and basolateral compartments for 40 min to stabilize physiological parameters.

HBSS is then removed from the apical compartment and replaced with test compound dosing solutions. The solutions are made by diluting 10 mM test compound in DMSO with HBSS to give a final test compound concentration of 10 μM (final DMSO concentration 1%). The fluorescent integrity marker lucifer yellow is also included in the dosing solution. Analytical standards are made from dosing solutions. Test compound permeability is assessed in duplicate. On each plate compounds of known permeability characteristics are run as controls.

The apical compartment inserts are then placed into ‘companion’ plates containing fresh HBSS. For basolateral to apical (B-A) permeability determination the experiment is initiated by replacing buffer in the inserts then placing them in companion plates containing dosing solutions. At 120 min the companion plate is removed and apical and basolateral samples diluted for analysis by LC-MS/MS. The starting concentration (C₀) and experimental recovery is calculated from both apical and basolateral compartment concentrations.

The integrity of the monolayers throughout the experiment is checked by monitoring lucifer yellow permeation using fluorimetric analysis. Lucifer yellow permeation is low if monolayers have not been damaged. Test and control compounds are quantified by LC-MS/MS cassette analysis using a 5-point calibration with appropriate dilution of the samples. Generic analytical conditions are used.

If a lucifer yellow Pₐₚₚ value is above QC limits in one individual test compound well, then an n=1 result is reported. If lucifer yellow Pₐₚₚ values are above QC limits in both replicate wells for a test compound, the compound is re-tested. Consistently high lucifer yellow permeation for a particular compound in both wells indicates toxicity. No further experiments are performed in this case.

### Data Analysis

The permeability coefficient for each compound (Pₐₚₚ) is calculated from the following equation:

$${P}_{app} = \frac{\frac{d Q}{d t}}{{C}_{0} \times A}$$

Where dQ/dt is the rate of permeation of the drug across the cells, C₀ is the donor compartment concentration at time zero and A is the area of the cell monolayer. C₀ is obtained from analysis of donor and receiver compartments at the end of the incubation period. It is assumed that all of the test compound measured after 120 min incubation was initially present in the donor compartment at 0 min. An asymmetry index (Al) is derived as follows:

$$Al = \frac{{P}_{app} ( B - A )}{{P}_{app} ( A - B )}$$

An asymmetry index above unity shows efflux from the Caco-2 cells, which indicates that the compound may have potential absorption problems in vivo.

The apparent permeability (Pₐₚₚ(A−B)) values of test compounds are compared to those of control compounds, atenolol and propranolol, that have human absorption of approximately 50 and 90% respectively (Zhao, Y. H., et al., (2001). Evaluation of Human Intestinal Absorption Data and Subsequent Derivation of a Quantitative Structure-Activity Relationship (QSAR) with the Abraham Descriptors. Journal of Pharmaceutical Sciences. 90 (6), 749-784). Talinolol (a known P-gp substrate (Deferme, S., Mols, R., Van Driessche, W., Augustijns, P. (2002). Apricot Extract Inhibits the P-gp-Mediated Efflux of Talinolol. Journal of Pharmaceutical Sciences. 91(12), 2539-48)) is also included as a control compound to assess whether functional P-gp is present in the Caco-2 cell monolayer.

### 4. Cytochrome P450 Inhibition (5 Isoform IC₅₀ Determination))

### Experimental Procedure

### CYP1A Inhibition

Six test compound concentrations (0.05, 0.25, 0.5, 2.5, 5, 25 μM in DMSO; final DMSO concentration=0.35%) are incubated with human liver microsomes (0.25 mg/ml) and NADPH (1 mM) in the presence of the probe substrate ethoxyresorufin (0.5 μM) for 5 min at 37° C. The selective CYP1A inhibitor, alpha-naphthoflavone, is screened alongside the test compounds as a positive control.

### CYP2C9 Inhibition

Six test compound concentrations (0.05, 0.25, 0.5, 2.5, 5, 25 μM in DMSO; final DMSO concentration=0.25%) are incubated with human liver microsomes (1 mg/ml) and NADPH (1 mM) in the presence of the probe substrate tolbutamide (120 μM) for 60 min at 37° C. The selective CYP2C9 inhibitor, sulphaphenazole, is screened alongside the test compounds as a positive control.

### CYP2C19 Inhibition

Six test compound concentrations (0.05, 0.25, 0.5, 2.5, 5, 25 μM in DMSO; final DMSO concentration=0.25%) are incubated with human liver microsomes (0.5 mg/ml) and NADPH (1 mM) in the presence of the probe substrate mephenyloin (25 μM) for 60 min at 37° C. The selective CYP2C19 inhibitor, tranylcypromine, is screened alongside the test compounds as a positive control.

### CYP2D6 Inhibition

Six test compound concentrations (0.05, 0.25, 0.5, 2.5, 5, 25 μM in DMSO; final DMSO concentration=0.25%) are incubated with human liver microsomes (0.5 mg/ml) and NADPH (1 mM) in the presence of the probe substrate dextromethorphane (5 μM) for 30 min at 37° C. The selective CYP2D6 inhibitor, quinidine, is screened alongside the test compounds as a positive control.

### CYP3A4 Inhibition

Six test compound concentrations (0.05, 0.25, 0.5, 2.5, 5, 25 μM in DMSO; final DMSO concentration 0.26%) are incubated with human liver microsomes (0.25 mg/ml) and NADPH (1 mM) in the presence of the probe substrate midazolam (2.5 μM) for 5 min at 37° C. The selective CYP3A4 inhibitor, ketoconazole, is screened alongside the test compounds as a positive control.

For the CYP1A incubations, the reactions are terminated by the addition of methanol, and the formation of the metabolite, resorufin, is monitored by fluorescence (excitation wavelength=535 nm, emission wavelength=595 nm). For the CYP2C9, CYP2C19, CYP2D6, and CYP3A4 incubations, the reactions are terminated by the addition of methanol containing internal standard. The samples are then centrifuged, and the supernatants are combined, for the simultaneous analysis of 4-hydroxytolbutamide, 4-hydroxymephenyloin, dextrorphan, and 1-hydroxymidazolam plus internal standard by LC-MS/MS. Generic LC-MS/MS conditions are used. Formic acid in deionised water (final concentration=0.1%) is added to the final sample prior to analysis. A decrease in the formation of the metabolites compared to vehicle control is used to calculate an IC₅₀ value (test compound concentration which produces 50% inhibition).

### 5. Plasma Protein Binding (10%)

### Experimental Procedure

Solutions of test compound (5 μM, 0.5% final DMSO concentration) are prepared in buffer (pH 7.4) and 10% plasma (v/v in buffer). The experiment is performed using equilibrium dialysis with the two compartments separated by a semi-permeable membrane. The buffer solution is added to one side of the membrane and the plasma solution to the other side. Standards are prepared in plasma and buffer and are incubated at 37° C. Corresponding solutions for each compound are analyzed in cassettes by LC-MS/MS.

### Quantitative Analysis

After equilibration, samples are taken from both sides of the membrane. The solutions for each batch of compounds are combined into two groups (plasma-free and plasma-containing) then cassette analyzed by LC-MS/MS using two sets of calibration standards for plasma-free (7 points) and plasma-containing solutions (6 points). Generic LC-MS/MS conditions are used. Samples are quantified using standard curves prepared in the equivalent matrix. The compounds are tested in duplicate.

A control compound is included in each experiment.

### Data Analysis

$$fu = \frac{1 - ( ( PC - PF ) )}{( PC )}$$

fu=fraction unbound
PC=sample concentration in protein containing side
PF=sample concentration in protein free side
fu at 10% plasma is converted to fu 100% plasma using the following equation:

$${fu}_{100 \%} = \frac{{fu}_{10 \%}}{10 - ( {9}^{*} {fu}_{10 \%} )}$$

### Examples of a Pharmaceutical Composition

As a specific embodiment of an oral composition of a compound of the present invention, 27 mg of Example 1 is formulated with sufficient finely divided lactose to provide a total amount of 580 to 590 mg to fill a size 0 hard gelatin capsule.

As another specific embodiment of an oral composition of a compound of the present invention, 33 mg of Example 22 is formulated with sufficient finely divided lactose to provide a total amount of 580 to 590 mg to fill a size 0 hard gelatin capsule.

While the invention has been described and illustrated in reference to certain preferred embodiments thereof, those skilled in the art will appreciate that various changes, modifications and substitutions can be made therein without departing from the spirit and scope of the invention. For example, effective dosages, other than the preferred doses as set forth above, may be applicable as a consequence of the specific pharmacological responses observed and may vary depending upon the particular active compound selected, as well as from the type of formulation and mode of administration employed, and such expected variations or differences in the results are contemplated in accordance with the objects and practices of the present invention. It is intended, therefore, that the invention be limited only by the scope of the claims which follow and that such claims be interpreted as broadly as is reasonable.

1. A compound according to formula (I) 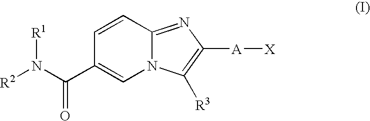
and enantiomers, diastereomers, tautomers, solvates and pharmaceutically acceptable salts thereof, wherein
R¹ and R² are independently from each other selected from
H,
C₁₋₆ alkyl,
C₁₋₆ alkylene-O—C₁₋₆alkyl
C₁₋₃ alkylene-heterocyclyl, and
C₁₋₆ alkylene-C₃₋₇cycloalkyl, or
R¹ and R², together with the nitrogen atom to which they are attached to, form a 5 to 6-membered ring which may additionally contain one oxygen atom in the ring and which is unsubstituted or substituted by one or more substituents selected from OH, C₁₋₆alkyl, O—C₁₋₆alkyl, C₀₋₃alkylene-C₃₋₅cycloalkyl, C₁₋₆alkyl-O—C₁₋₆alkyl and (CH₂)₀₋₃-phenyl;
A is —NH—,
—C₁₋₆alkylene,
—C₂₋₆alkenylene,
—C₂₋₆alkinylene or
a bond
wherein alkylene, alkenylene and alkinylene are unsubstituted or substituted with one or more R⁷;
R⁷ is independently selected from
C₁₋₆alkyl,
OR¹⁴,
NR^{15a}R^{15b},
halogen,
phenyl and
heteroaryl,
wherein phenyl and heteroaryl are unsubstituted or substituted by 1 to 3 R^{4a};
X is
CN,
C₃₋₈cycloalkyl, unsubstituted or substituted with one or more halogen atoms,
4 to 8-membered saturated or unsaturated heterocyclyl containing 3 or 4 heteroatoms independently selected from N, O and S,
5- to 6-membered heteroaryl containing 3 or 4 heteroatoms independently selected from N, O and S,
5- to 6-membered heteroaryl containing 1 to 3 heteroatoms independently selected from N, O and S, where the heteroaryl ring is fused with a 4 to 8-membered saturated or unsaturated heterocyclyl containing 1 to 3 heteroatoms independently selected from N, O and S or fused with a 5- to 6-membered heteroaryl containing 1 to 3 heteroatoms independently selected from N, O and S,
—C(O)—R⁶,
—OR¹⁴,
halogen or
NR^{15a}R^{15b},
wherein each heterocyclyl or heteroaryl is unsubstituted or substituted by 1 to 3 R^{4a} and/or 1 R^{4b} and/or 1 R⁵;
R^{4a} is halogen,
CN,
C₁₋₆alkyl, unsubstituted or substituted with one or more substituents selected from halogen atoms, C₁₋₆alkyl, O—C₁₋₆alkyl and OH,
O—C₁₋₆alkyl, wherein alkyl is unsubstituted or substituted with one or more substituents selected from halogen atoms and OH,
C₃₋₈cycloalkyl, unsubstituted or substituted with one or more substituents selected from halogen atoms and OH, or
OH;
R^{4b} is
C(O)NH₂,
C(O)NH—C₁₋₆alkyl,
C(O)N—(C₁₋₆alkyl)₂,
SO₂—C₁₋₆alkyl,
C(O)NH—SO₂—C₁₋₆alkyl,
oxo, whereby the ring is at least partially saturated,
NH₂,
N—(C₁₋₆alkyl)₂,
NH—SO₂—CH₃, or
NH—SO₂—CF₃;
R⁵ is 5 to 6-membered saturated or unsaturated heterocyclyl containing 1 to 3 heteroatoms independently selected from N, O and S
wherein heterocyclyl is unsubstituted or substituted by 1 or 2 R^{4a};
R⁶ is
OH,
O—C₁₋₆alkyl, wherein alkyl is unsubstituted or substituted with one or more R¹⁶,
4- to 8-membered heterocyclyl containing 1 to 3 heteroatoms independently selected from N, O and S, or
NR^{16a}R^{16b}
wherein heterocyclyl is unsubstituted or substituted by 1 or 2 R^{4a};
R³ is —(CR⁸R⁹)ₙ-T;
R⁸ and R⁹ are independently from each other selected from
H,
OH,
halogen,
C₁₋₆alkyl, and
n is 1 to 6;
T is 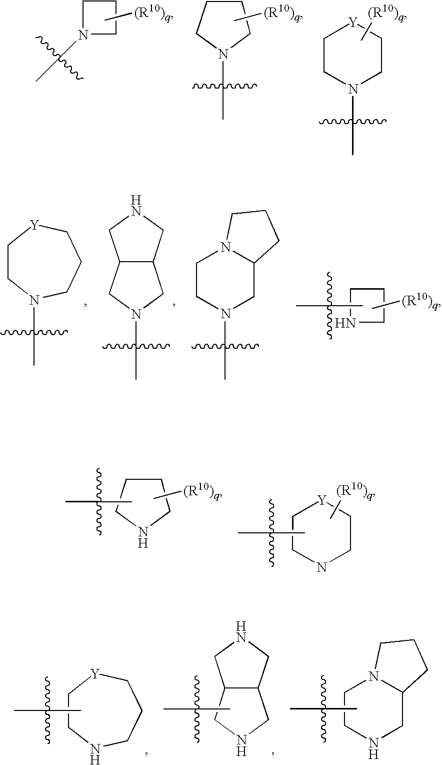
or NR¹²R¹³;
R¹⁰ is
H,
NH₂,
OH,
C₁₋₆alkyl,
halogen,
NH(C₁₋₆alkyl),
N(C₁₋₆alkyl)₂,
phenyl or
heteroaryl,
wherein phenyl and heteroaryl are unsubstituted or substituted by 1 to 3 R^{4a};
q is 1 or 2;
Y is
CH₂,
NR¹¹ or
O;
R¹¹ is
H,
C₁₋₆alkyl or
(CH₂)₀₋₆—C₃₋₇cycloalkyl;
R¹² and R¹³ are independently from each other selected from
H,
C₁₋₆ alkyl,
(CH₂)₀₋₂—C₃₋₇cycloalkyl and
C₁₋₆alkylene-O—C₁₋₆alkyl;
wherein alkyl, alkylene and cycloalkyl are unsubstituted or substituted by 1 to 3 R^{4a}.
R¹⁴ is
H
C₁₋₆alkyl, unsubstituted or substituted with one or more substituents selected from halogen,
phenyl or
heteroaryl,
wherein phenyl and heteroaryl are unsubstituted or substituted by 1 to 3 R^{4a};
R^{15a} and R^{15b} are independently from each other selected from
H,
C₁₋₆alkyl, unsubstituted or substituted with one or more substituents selected from halogen, OH, O(C₁₋₆alkyl), NH₂, NH(C₁₋₆alkyl) and N(C₁₋₆ alkyl)₂,
C(O)C₁₋₆alkyl,
C(O)OC₁₋₆alkyl,
phenyl,
heteroaryl and
phenyl fused with a 5- to 6-membered saturated or unsaturated heterocyclyl containing 1 to 3 heteroatoms independently selected from N, O and S, or fused with a 5- to 6-membered heteroaryl containing 1 to 3 heteroatoms independently selected from N, O and S,
wherein each phenyl, heterocyclyl and heteroaryl is unsubstituted or substituted by 1 to 3 R^{4a};
R¹⁶, R^{16a} and R^{16b} are independently from each other selected from
H,
C₁₋₆alkyl, unsubstituted or substituted with one or more substituents selected from halogen, OH, O(C₁₋₆alkyl), NH₂, NH(C₁₋₆alkyl) and N(C₁₋₆ alkyl)₂,
C₀₋₃alkylene-C₃₋₅cycloalkyl,
phenyl and
heteroaryl,
wherein phenyl and heteroaryl are unsubstituted or substituted by 1 to 3 R^{4a}.

2. The compound according to claim 1, wherein
R¹ and R² are independently from each other selected from
H,
C₁₋₆ alkyl,
C₁₋₆ alkylene-O—C₁₋₆alkyl
C₁₋₃ alkylene-heterocyclyl, and
C₁₋₆ alkylene-C₃₋₇cycloalkyl, or
R¹ and R², together with the nitrogen atom to which they are attached to, form a 5 to 6-membered ring which may additionally contain one oxygen atom in the ring and which is unsubstituted or substituted by one or more substituents selected from OH, C₁₋₆alkyl, O—C₁₋₆alkyl, C₀₋₃alkylene-C₃₋₅cycloalkyl, C₁₋₆alkyl-O—C₁₋₆alkyl and (CH₂)₀₋₃-phenyl;
A is
—NH—,
—C₁₋₆alkylene,
—C₂₋₆alkenylene,
—C₂₋₆alkinylene or
a bond
wherein alkylene, alkenylene and alkinylene are unsubstituted or substituted with one or more R⁷;
R⁷ is independently selected from
C₁₋₆alkyl,
OR¹⁴,
NR^{15a}R^{15b},
halogen,
phenyl and
heteroaryl,
wherein phenyl and heteroaryl are unsubstituted or substituted by 1 to 3 R^{4a};
X is
CN,
C₃₋₈cycloalkyl, unsubstituted or substituted with one or more halogen atoms,
4 to 8-membered saturated or unsaturated heterocyclyl containing 3 or 4 heteroatoms independently selected from N, O and S,
5- to 6-membered heteroaryl containing 3 or 4 heteroatoms independently selected from N, O and S,
—C(O)—R⁶,
—OR¹⁴,
halogen or
NR^{15a}R^{15b},
wherein each heterocyclyl or heteroaryl is unsubstituted or substituted by 1 to 3 R^{4a} and/or 1 R^{4b} and/or 1 R⁵;
R^{4a} is
halogen,
CN,
C₁₋₆alkyl, unsubstituted or substituted with one or more halogen atoms, O—C₁₋₆alkyl, wherein alkyl is unsubstituted or substituted with one or more halogen atoms, or
OH;
R^{4b} is
C(O)NH₂,
C(O)NH—C₁₋₆alkyl,
C(O)N—(C₁₋₆alkyl)₂,
SO₂—C₁₋₆alkyl,
C(O)NH—SO₂—C₁₋₆alkyl,
oxo, whereby the ring is at least partially saturated,
NH₂,
NH—C₁₋₆alkyl,
N—(C₁₋₆alkyl)₂,
NH—SO₂—CH₃, or
NH—SO₂—CF₃;
R⁵ is 5 to 6-membered saturated or unsaturated heterocyclyl containing 1 to 3 heteroatoms independently selected from N, O and S
wherein heterocyclyl is unsubstituted or substituted by 1 or 2 R^{aa};
R⁶ is
OH,
O—C₁₋₆alkyl, wherein alkyl is unsubstituted or substituted with one or more R¹⁶, or NR^{16a}R^{16b};
R³ is —(CR⁸R⁹)ₙ-T;
R⁸ and R⁹ are independently from each other selected from
H,
OH,
halogen,
C₁₋₆alkyl, and
O—C₁₋₆alkyl,
n is 1 to 6;
T is 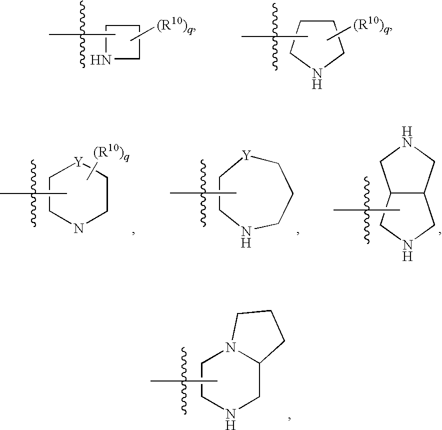
or NR¹²R¹³;
R¹⁰ is
H,
NH₂;
OH,
C₁₋₆alkyl,
halogen,
NH(C₁₋₆alkyl),
N(C₁₋₆alkyl)₂,
phenyl or
heteroaryl,
wherein phenyl and heteroaryl are unsubstituted or substituted by 1 to 3 R^{4a};
q is 1 or 2;
Y is
CH₂,
NR¹¹ or
O;
R¹¹ is
H,
C₁₋₆alkyl or
(CH₂)₀₋₆—C₃₋₇cycloalkyl;
R¹² and R¹³ are independently from each other selected from
H,
C₁₋₆ alkyl,
(CH₂)₀₋₂—C₃₋₇cycloalkyl and
C₁₋₆alkylene-O—C₁₋₆alkyl;
wherein alkyl, alkylene and cycloalkyl are unsubstituted or substituted by 1 to 3 R^{4a},
R¹⁴ is
H,
C₁₋₆alkyl, unsubstituted or substituted with one or more substituents selected from
halogen,
phenyl or
heteroaryl,
wherein phenyl and heteroaryl are unsubstituted or substituted by 1 to 3 R^{4a};
R^{15a} and R^{15b} are independently from each other selected from
H,
C₁₋₆alkyl, unsubstituted or substituted with one or more substituents selected from halogen, OH, O(C₁₋₆alkyl), NH₂, NH(C₁₋₆alkyl) and N(C₁₋₆ alkyl)₂,
phenyl and
heteroaryl,
wherein phenyl and heteroaryl are unsubstituted or substituted by 1 to 3 R^{4a}, and
C(O)C₁₋₆alkyl;
R¹⁶, R^{16a} and R^{16b} are independently from each other selected from
H,
C₁₋₆alkyl, unsubstituted or substituted with one or more substituents selected from halogen, OH, O(C₁₋₆alkyl), NH₂, NH(C₁₋₆alkyl) and N(C₁₋₆ alkyl)₂,
C₀₋₃alkylene-C₃₋₅cycloalkyl,
phenyl and
heteroaryl,
wherein phenyl and heteroaryl are unsubstituted or substituted by 1 to 3 R^{4a}.

3. The compound of claim 1 wherein A is —NH— or a bond.

4. The compound of claim 1 wherein R¹ and R² are independently from each other C₃₋₆alkyl or R¹ and R² form together with the nitrogen atom to which they are attached to a 5 to 6-membered ring which may additionally contain one oxygen atom in the ring and which is unsubstituted or substituted by one or more substituents selected from OH, C₁₋₆alkyl, C₀₋₃alkylene-C₃₋₅cycloalkyl, O—C₁₋₆alkyl, C₁₋₆alkyl-O—C₁₋₆alkyl and (CH₂)₀₋₃-phenyl.

5. The compound of claim 1 wherein T is NR¹²R¹³.

6. The compound of claim 5 wherein R¹² and R¹³ are independently from each other selected from H, C₁₋₃alkyl and (CH₂)₀₋₂—C₃₋₆cycloalkyl, wherein alkyl and cycloalkyl are unsubstituted or substituted by 1 to 3 R^{4a}.

7. The compound of claim 1 wherein T is selected from

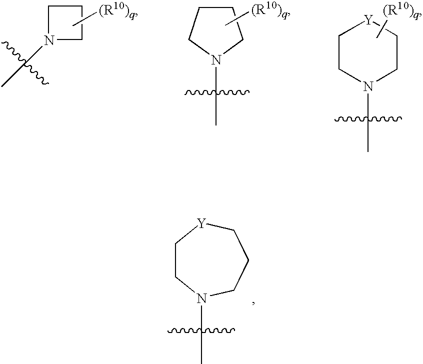

8. The compound of claim 7 wherein Y is CH₂ or NR¹¹ and R¹⁰ is H, NH₂, C₁₋₆alkyl, NH(C₁₋₆alkyl) or N(C₁₋₆alkyl)₂.

9. The compound of claim 1 wherein
X is
4 to 8-membered saturated or unsaturated heterocyclyl containing 3 or 4 heteroatoms independently selected from N, O and S, or
5- to 6-membered heteroaryl containing 3 or 4 heteroatoms independently selected from N, O and S,
wherein each heterocyclyl or heteroaryl is unsubstituted or substituted by 1 to 3 R^{4a} and/or 1 R^{4b} and/or 1 R⁵.

10. The compound of claim 1 wherein
X is
—C(O)—R⁶,
—OR¹⁴, or
—NR^{15a}R^{15b}.

11. The compound of claim 10, wherein R⁶ is —O—C₁₋₆alkyl, wherein alkyl is unsubstituted or substituted with one or more R¹⁶.

12. The compound of claim 10, wherein R⁶ is NR^{16a}R^{16b}.

13. The compound of claim 12, wherein R⁶ is NH—C₁₋₆alkyl, wherein alkyl is unsubstituted or substituted with one or more R¹⁶.

14. The compound of claim 1, wherein the compound is formulated as a medicament.

15. The compound of claim 1, wherein the compound is formulated as a melanocortin-4 receptor antagonist.

16. The compound of claim 1, wherein the compound is formulated for the prophylaxis or treatment of disorders, diseases or conditions responsive to the inactivation of the melanocortin-4 receptor in a mammal.

17. The compound of claim 16, wherein the compound is formulated for the prophylaxis or treatment of cachexia.

18. The compound of claim 17, wherein the compound is formulated for the prophylaxis or treatment of cachexia selected from cancer cachexia, cachexia induced by chronic kidney disease (CKD) or cachexia induced by chronic heart failure (CHF).

19. The compound of claim 16, wherein the compound is formulated for the prophylaxis or treatment of muscle wasting.

20. The compound of claim 16, wherein the compound is formulated for the prophylaxis or treatment of anorexia.

21. The compound of claim 20 wherein the compound is formulated for the prophylaxis or treatment of anorexia selected from anorexia nervosa or anorexia induced by radiotherapy or chemotherapy.

22. The compound of claim 16, wherein the compound is formulated for the prophylaxis or treatment of anxiety and/or depression.

23. The compound of claim 16, wherein the compound is formulated for the prophylaxis or treatment of amyotrophic lateral sclerosis (ALS).

24. The compound of claim 16, wherein the compound is formulated for the prophylaxis or treatment of pain and neuropathic pain.

25. A method of using the compound of claim 1, comprising:
formulating the compound as a medicament for the prophylaxis or treatment of disorders, diseases or conditions responsive to the inactivation of the melanocortin-4 receptor in a mammal; and
administering a pharmaceutically suitable form of the medicament to a mammal.

26. The method according to claim 25, wherein the compound is formulated and administered as a medicament for the prophylaxis or treatment of cachexia.

27. The method according to claim 26, wherein, wherein the compound is formulated and administered as a medicament for the prophylaxis or treatment of cachexia induced by cancer, chronic kidney disease (CKD) or chronic heart failure (CHF).

28. The method according to claim 25, wherein the compound is formulated and administered as a medicament for the prophylaxis or treatment of muscle wasting.

29. The method according to claim 25, wherein the compound is formulated and administered as a medicament for the prophylaxis or treatment of anorexia.

30. The method according to claim 29, wherein the compound is formulated and administered as a medicament for the prophylaxis or treatment of anorexia selected from anorexia nervosa or anorexia induced by radiotherapy or chemotherapy.

31. The method according to claim 25, wherein the compound is formulated and administered as a medicament for the prophylaxis or treatment of anxiety and/or depression.

32. The method according to claim 25, wherein the compound is formulated and administered as a medicament for the prophylaxis or treatment of amyotrophic lateral sclerosis (ALS).

33. The method according to claim 25, wherein the compound is formulated and administered as a medicament for the prophylaxis or treatment of pain and neuropathic pain.

34. A pharmaceutical composition comprising a compound of claim 1 and a pharmaceutically acceptable carrier.
